(19) United States
(12) Patent Application Publication
(10) Pub. No.: US 2017/0246468 A1
(43) Pub. Date: Aug. 31, 2017

(54) METHODS AND SYSTEMS FOR CONTROLLING OR TUNING THE ELECTRIC FIELD GENERATED IN SKIN OR TISSUE DURING COLD PLASMA SKIN TREATMENTS

(71) Applicant: EP Technologies LLC, Akron, OH (US)

(72) Inventors: Sameer Kalghatgi, Copley, OH (US); Tsung-Chan Tsai, Cuyahoga Falls, OH (US); Jeffrey S. Louis, Akron, OH (US); Robert L. Gray, Kent, OH (US)

(21) Appl. No.: 15/441,371

(22) Filed: Feb. 24, 2017

Related U.S. Application Data
(60) Provisional application No. 62/299,783, filed on Feb. 25, 2016.

(51) Int. Cl. A61N 1/44

(52) U.S. Cl. CPC A61N 1/44

(57) ABSTRACT

Exemplary systems and methods associated with skin treatments using non-thermal plasma to porate skin or tissues using conductive elements to direct an electric field through targeted regions of the skin or tissue. Various configurations of conductive elements and associated circuitry may be used to tune and control the electric field.

### RELATED APPLICATIONS

This application claims the benefit of U.S. Provisional Application No. 62/299,783, filed Feb. 25, 2016, incorporated fully herein by reference.

### TECHNICAL FIELD

The present invention relates generally to methods and systems for enabling or enhancing skin treatments using non-thermal plasma, and more particularly for controlling and tuning the process of opening pores in skin or tissue, including, for example, for transporting one or more substances across layers of skin or tissue for deep tissue sanitization, delivery of vaccines, drugs and cosmetics, improvement of skin health, and the like.

### BACKGROUND OF THE INVENTION

Transdermal delivery of a treatment substance is localized, non-invasive, and has the potential for sustained and controlled release of various substances, including, for example, drugs and other molecules. In addition, transdermal delivery avoids first-pass metabolism, which reduces the concentration of certain substances before the substance reaches the circulatory system. In addition, percutaneous absorption can minimize the risk of irritation of the gastrointestinal tract and minimize pain and other complications associated with parenteral administration.

Transdermal delivery, however, requires molecules to pass through the skin. The outer layer of the skin is the stratum corneum (“SC”). The SC is composed of dead, flattened, keratin-rich cells, called corneocytes. These dense cells are surrounded by a complex mixture of intercellular lipids—namely, ceramides, free fatty acids, cholesterol and cholesterol sulfate. The predominant diffusional path for a molecule crossing the SC appears to be intercellular. The remaining layers of the skin are the epidermis (viable epidermis), the dermis, and the subcutaneous tissue.

Only a small percentage of substances or compounds can be delivered transdermally because skin has barrier properties, namely the highly lipophilic SC, that prevents molecules from penetrating the skin. As a result, only, molecules with a molecular weight (MW) of less than 500 Dalton can be administered topically or percutaneously. Often, for pharmaceutical applications, the development of innovative compounds is restricted to a MW of less than 500 Dalton when topical dermatological therapy, percutaneous systemic therapy or vaccination is the objective. In addition, transport of most drugs across the skin is very slow, and lag times to reach steady-state fluxes are measured in hours. Achievement of a therapeutically effective drug level is therefore difficult without artificially enhancing skin permeation.

A number of chemical and physical enhancement techniques have been developed in an attempt to compromise the skin barrier function in a reversible manner. These attempts may be classified as passive and active methods.

Passive methods for enhancing transdermal drug delivery include the use of vehicles such as ointments, creams, gels and passive patch technology. In addition, there are other passive methods that artificially damage the barrier in order to allow improved permeation of active substances, such as, for example, micro-needles that produce small holes having a depth of approximately 100-200 μm in the skin to allow improved permeation. The amount of substance that can be delivered using these methods is limited because the barrier properties of the skin are not fundamentally changed.

Active methods for enhancing transdermal drug delivery systems involve the use of external energy to act as a driving force and/or act to reduce the SC barrier resistance and enhance permeation of drug molecules into the skin. Iontophoresis and electroporation are two common methods of active transdermal drug delivery systems.

Iontophoresis is the process of increasing the permeation of charged or polar drugs into skin by the application of an electric current. The amount of a compound delivered is directly proportional to the quantity of charge passed; i.e., it depends on the applied current, the duration of current application and the surface area of the skin in contact with the active electrode compartment. Advantages of iontophoresis include an improved onset time and also a more rapid offset time—that is, once the current is switched off, there is no further transportation of the compound.

To deliver drugs using iontophoresis, a drug is applied under an electrode of the same charge as the drug and return electrode having an opposite charge is placed on the body surface. A current below the level of the patient's pain threshold is applied for an appropriate length of time. Because like charges repel one another, the electrical current increases the permeation of the drug into surface tissues, without altering the structure of the SC. Iontophoresis transports drugs primarily through existing pathways in skin, such as hair follicles and sweat glands. Iontophoresis is typically used when a low level delivery is desired over a long time period. Iontophoresis involves the use of relatively low transdermal voltages (<100 V).

Transdermal absorption of drugs through iontophoresis is affected by drug concentration, polarity of drugs, pH of donor solution, ionic competition, ionic strength, electrode polarity, etc. Iontophoresis has safety concerns due to the use of electrical contacts on the skin, which may result in patient discomfort, muscle contraction, pain and, sometimes, even skin damage and burns.

Electroporation is a method for transdermal drug delivery that consists of applying high-voltage pulses to skin. The applied high-voltage plays a dual role. First, it creates new pathways for enhancing drug permeability and second, it provides an electrical force for driving like charged molecules through the newly created pores. Electroporation is usually used on the unilamellar phospholipid bilayers of cell membranes. However, it has been demonstrated that electroporation of skin is feasible, even though the SC contains multilamellar, intercellular lipid bilayers with phospholipids and no living cells.

Electroporation of skin requires high transdermal voltages (˜100 V or more, usually >100 V). In transdermal electroporation, the predominant voltage drop of an applied electric pulse to the skin develops across the SC. This voltage distribution causes electric breakdown (electroporation) of the SC. If the voltage of the applied pulses exceeds a voltage threshold of about 75 to 100 V, micro channels or “local transport regions” are created through the breakdown sites of the SC.

DNA introduction is the most common use for electroporation. Electroporation of isolated cells has also been used for (1) introduction of enzymes, antibodies, and other biochemical reagents for intracellular assays; (2) selective biochemical loading of one size cell in the presence of many smaller cells; (3) introduction of virus and other particles; (4) cell killing under nontoxic conditions; and (5) insertion of membrane macromolecules into the cell membrane.

The presence of electrodes in contact with skin/tissue and the delivery of current into skin/tissue in this manner leads to patient discomfort, muscle contractions, pain and, sometimes, even skin damage and burns. In addition, electroporation often takes hours, e.g., 6 to 24 hours, to drive therapeutic amount of drugs or other molecules transdermally.

U.S. Pat. No. 8,455,228, entitled “Method to Facilitate Directed Delivery and Electroporation Using a Charged Steam,” state that “the method and apparatus in accordance with the present invention are effective in using an electrical field to adjust the electrochemical potential of a target molecule thereby providing molecular transport of the target molecule into and/or across the tissue by a diffusive transport mechanism.” The '228 patent discloses a first embodiment with dielectric properties to assure that it will hold a charge sufficient to polarize charged entities contained within a vessel and a plurality of electroporation applicators. The process described in the '228 patent disclosure suffers from several deficiencies. First, it requires molecules that may be polarized or charged, second it requires electroporation applicators, and third, the molecule is contacted with plasma during the process, which may irreversible modify the molecular structure leading to adverse results. In addition it is well known that interaction of molecules with plasma leads to the oxidation of such molecules.

The '228 patent also discloses a second embodiment utilizing a plasma jet with a ground ring around an inner chamber. The disclosure related to this device includes containing cells suspended in fluid in the inner chamber and promoting uptake into the cells; or injecting plasmid intradermally and exposing the injection site to plasma.

U.S. patent publication No. 2014/0188071 discloses a method of applying a substance to the skin and applying plasma to the same area. The '071 publication discloses an open cell foam to hold a drug, water, etc., and applies plasma through the open cell foam. Applying plasma through the open cell foam and contacting the drugs with plasma may alter the molecular structure of the drugs and cause undesirable side effects and/or render the drug ineffective.

U.S. patent publication 2012/0288934 discloses a plasma jet and the active substance is applied to the skin with the gas stream of the plasma jet and is transported onto the region of the living cells through the barrier door that has been opened by the plasma. Applying the active substance with the gas stream of the plasma jet may alter the molecular structure of the active substance and cause undesirable side effects and/or render the active substance ineffective.

### SUMMARY

According to one aspect of the present invention, an apparatus for controlling an electric field associated with a skin treatment includes a plasma generating device for generating a plasma, a power supply for powering the plasma generating device, first circuitry for providing one or more first electrical pulses to the plasma generating device, and a first conductive element in contact with the skin for affecting the electric field associated with the generated plasma, where at least a portion of the electric field is directed through a first region of the skin by the plasma and the first conductive element.

The descriptions of the invention do not limit the words used in the claims in any way or the scope of the claims or invention. The words used in the claims have all of their full ordinary meanings.

### BRIEF DESCRIPTION OF THE DRAWINGS

In the accompanying drawings, which are incorporated in and constitute a part of the specification, embodiments of the invention are illustrated, which, together with a general description of the invention given above, and the detailed description given below, serve to exemplify embodiments of this invention.

FIG. 1 illustrates a cross section of an exemplary skin treatment apparatus that includes an exemplary transdermal delivery system.

FIG. 2A illustrates a cross-section view of an exemplary skin treatment apparatus that includes an exemplary transdermal delivery system and depictions of the electric field associated with the generated plasma without a conductive element.

FIG. 2B illustrates a cross-section view of an exemplary skin treatment apparatus that includes an exemplary transdermal delivery system and depictions of the electric field associated with the generated plasma with a grounded conductive element.

FIG. 3A illustrates an exemplary transdermal delivery system without a conductive element depicting the associated respective electric field within a detailed cross-section view of skin.

FIG. 3B illustrates an exemplary transdermal delivery system with a conductive element depicting the associated respective electric field within a detailed cross-section view of skin.

FIG. 4A is a cross-section drawing of cells of a skin model exposed to a plasma.

FIG. 4B shows field lines within the skin cells shown in FIG. 4A, as the electric field is directed through these targeted skin cells.

FIG. 4C shows the electric field within the skin cells shown in FIG. 4A, as the electric field is directed through these targeted skin cells.

FIG. 5 is block diagram of an exemplary embodiment of a skin treatment apparatus for controlling an electric field associated with a skin treatment.

FIG. 6 shows one embodiment of a skin treatment apparatus with separate components.

FIG. 7 shows a skin treatment apparatus with integrated components.

FIG. 8 shows another skin treatment apparatus with integrated components.

FIG. 9 shows another skin treatment apparatus with integrated components.

FIG. 10A illustrates a cross-section of an exemplary skin treatment system with a non-thermal DBD generator.

FIG. 10B illustrates a cross-section of another exemplary skin treatment system with a non-thermal DBD generator.

FIG. 11A illustrates a cross-section of an exemplary skin treatment system with a floating-electrode DBD plasma generator that generates a plasma jet.

FIG. 11B illustrates a cross-section of another exemplary skin treatment system with a floating-electrode DBD plasma generator that generates a plasma jet.

FIG. 12 illustrates a cross-section of an exemplary skin treatment system with a plasma generating device and a ring-shaped conductive element.

FIG. 13 illustrates a cross-section of another exemplary skin treatment system with a plasma generating device and a ring-shaped conductive element.

FIG. 14 illustrates a cross-section of another exemplary skin treatment system with a plasma generating device and a ring-shaped conductive element.

FIG. 15A illustrates a cross-section of an exemplary skin treatment system in a non-ready position.

FIG. 15B illustrates a cross-section of an exemplary skin treatment system in a ready position.

FIG. 15C illustrates a cross-section of an exemplary skin treatment system generating plasma.

FIG. 16A shows an exemplary single conductive element connected to ground.

FIG. 16B shows an exemplary segmented conductive element with eight evenly spaced and same-size segments connected to ground.

FIG. 16C shows an exemplary segmented conductive element with four evenly spaced and same-size segments connected to ground.

FIG. 16D shows an exemplary segmented conductive element with two evenly spaced and same-size segments connected to ground.

FIG. 17A shows plasma generated above skin without any conductive element.

FIG. 17B shows the electric field at the top of the skin.

FIG. 17C shows a cross-section of the electric field throughout the skin.

FIG. 18A shows an exemplary conductive element surrounding a plasma.

FIG. 18B shows the electric field at the top of the skin.

FIG. 18C shows a cross-section of the electric field throughout the skin.

FIG. 19A shows an exemplary conductive element surrounding a plasma.

FIG. 19B shows the electric field at the top of the skin.

FIG. 19C shows a cross-section of the electric field throughout the skin.

FIG. 20A shows an exemplary conductive element surrounding a plasma.

FIG. 20B shows the electric field at the top of the skin.

FIG. 20C shows a cross-section of the electric field throughout the skin.

FIG. 21A shows an exemplary conductive element surrounding a plasma.

FIG. 21B shows the electric field at the top of the skin.

FIG. 21C shows a cross-section of the electric field throughout the skin.

FIG. 22A shows an exemplary conductive element surrounding a plasma.

FIG. 22B shows the electric field at the top of the skin.

FIG. 22C shows a cross-section of the electric field throughout the skin.

FIG. 23A shows an exemplary single conductive element connected to ground via a resistor.

FIG. 23B shows an exemplary segmented conductive element with eight evenly spaced and same-size segments connected to ground via a resistor.

FIG. 23C shows an exemplary segmented conductive element with four evenly spaced and same-size segments connected to ground via a resistor.

FIG. 23D shows an exemplary segmented conductive element with two evenly spaced and same-size segments connected to ground via a resistor.

FIG. 24A shows an exemplary current plot without a resistor.

FIG. 24B shows an exemplary current plot with an exemplary resistor value.

FIG. 24C shows another exemplary current plot with another exemplary resistor value.

FIG. 25A shows an exemplary single conductive element connected to ground via a resistor and a capacitor.

FIG. 25B shows an exemplary segmented conductive element with eight evenly spaced and same-size segments connected to ground via a resistor and a capacitor.

FIG. 25C shows an exemplary segmented conductive element with four evenly spaced and same-size segments connected to ground via a resistor and a capacitor.

FIG. 25D shows an exemplary segmented conductive element with two evenly spaced and same-size segments connected to ground via a resistor and a capacitor.

FIG. 26A shows an exemplary voltage plot with an exemplary capacitor value.

FIG. 26B shows another exemplary current plot with another exemplary capacitor value.

FIG. 27A shows an exemplary single conductive element connected to ground via a resistor and an inductor.

FIG. 27B shows an exemplary segmented conductive element with eight evenly spaced and same-size segments connected to ground via a resistor and an inductor.

FIG. 27C shows an exemplary segmented conductive element with four evenly spaced and same-size segments connected to ground via a resistor and an inductor.

FIG. 27D shows an exemplary segmented conductive element with two evenly spaced and same-size segments connected to ground via a resistor and an inductor.

FIG. 28A shows an exemplary capacitor time-based voltage/current characteristic when the plasma is on.

FIG. 28B shows an exemplary capacitor time-based voltage/current characteristic when the plasma is off.

FIG. 29A shows an exemplary inductor time-based voltage/current characteristic when the plasma is on.

FIG. 29B shows an exemplary inductor L time-based voltage/current characteristics when the plasma is off.

FIG. 30A shows an exemplary single conductive element connected to a bias voltage.

FIG. 30B shows an exemplary segmented conductive element with eight evenly spaced and same-size segments connected to ground or to a bias voltage.

FIG. 30C shows an exemplary segmented conductive element with four evenly spaced and same-size segments connected to ground or to a bias voltage.

FIG. 30D shows an exemplary segmented conductive element with two evenly spaced and same-size segments connected to ground or to a bias voltage.

FIG. 31A is a drawing of an exemplary transdermal delivery apparatus.

FIG. 31B is a cross-section drawing of the lower portion of the apparatus in FIG. 31A.

FIG. 32A is a drawing of another exemplary transdermal delivery apparatus.

FIG. 32B is a cross-section drawing of the lower portion of the apparatus in FIG. 32A.

FIG. 33 shows an exemplary method of treating skin with plasma.

FIG. 34 shows an exemplary method of treating skin with plasma and a treatment substance.

FIG. 35 shows another exemplary method of treating skin with plasma and a treatment substance.

FIG. 36 shows an exemplary method of treating skin with plasma using a conductive patch.

FIG. 37 shows an exemplary method of treating skin with an exemplary variable skin treatment apparatus.

FIG. 38 shows an exemplary method of tuning the characteristics of an electric field associated with a plasma skin treatment.

### DESCRIPTION

The following includes definitions of exemplary terms used throughout the disclosure. Both singular and plural forms of all terms fall within each meaning:

“Circuit” or “circuitry,” as used herein includes, but is not limited to, hardware, firmware, software or combinations of each to perform a function(s) or an action(s). For example, based on a desired feature or need, a circuit may include a software controlled microprocessor, discrete logic such as an application specific integrated circuit (ASIC), or other programmed logic device. A circuit may also be fully embodied as software. As used herein, “circuit” is considered synonymous with “logic.”

“Controller,” as used herein includes, but is not limited to, any circuit or device that coordinates and controls the operation of one or more input or output devices. For example, a controller can include a device having one or more processors, microprocessors, or central processing units (CPUs) capable of being programmed to perform input or output functions.

“Logic,” as used herein includes, but is not limited to, hardware, firmware, software or combinations of each to perform a function(s) or an action(s), or to cause a function or action from another component. For example, based on a desired application or need, logic may include a software controlled microprocessor, discrete logic such as an application specific integrated circuit (ASIC), or other programmed logic device. Logic may also be fully embodied as software. As used herein, “logic” is considered synonymous with “circuit.”

“Operative communication” or “circuit communication,” as used herein includes, but is not limited to, a communicative relationship between devices, logic, or circuits, including mechanical and pneumatic relationships. Direct electrical, electromagnetic, and optical connections and indirect electrical, electromagnetic, and optical connections are examples of such communications. Linkages, gears, chains, push rods, cams, keys, attaching hardware, and other components facilitating mechanical connections are also examples of such communications. Pneumatic devices and interconnecting pneumatic tubing may also contribute to operative communications. Two devices are in operative communication if an action from one causes an effect in the other, regardless of whether the action is modified by some other device. For example, two devices separated by one or more of the following: i) amplifiers, ii) filters, iii) transformers, iv) optical isolators, v) digital or analog buffers, vi) analog integrators, vii) other electronic circuitry, viii) fiber optic transceivers, ix) Bluetooth communications links, x) 802.11 communications links, xi) satellite communication links, xii) near-field communication, and xiii) other wireless communication links. As another example, an electromagnetic sensor is in operative communication with a signal if it receives electromagnetic radiation from the signal. As a final example, two devices not directly connected to each other, but both capable of interfacing with a third device, e.g., a central processing unit (CPU), are in operative communication.

“Processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random access memory (RAM), read-only memory (ROM), programmable read-only memory (PROM), erasable programmable read-only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

“Signal,” as used herein includes, but is not limited to, one or more electrical signals, including analog or digital signals, one or more computer instructions, a bit or bit stream, or the like.

“Software,” as used herein includes, but is not limited to, one or more computer readable or executable instructions that cause a computer or other electronic device to perform functions, actions, or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system, or other types of executable instructions. It will be appreciated by one of ordinary skill in the art that the form of software is dependent on, for example, requirements of a desired application, the environment it runs on, or the desires of a designer/programmer or the like.

While the above exemplary definitions have been provided, it is Applicant's intention that the broadest reasonable interpretation consistent with this specification be used for these and other terms.

Applicants have developed techniques for skin treatments that can include moving substances, molecules, drugs, DNA and the like across layers of the skin, both intercellularly (between cells) and intracellularly (into the cells) using plasma. Applicants filed U.S. Provisional Application Ser. No. 61/883,701 filed on Sep. 27, 2013 and U.S. Non-Provisional application Ser. No. 14/500,144, filed on Sep. 29, 2014, both of which are entitled Method and Apparatus for Delivery of Molecules Across Layers of the Skin, and both are incorporated herein by reference in their entirety. Applicants' exemplary methods utilize plasma for providing a safe, contact-less delivery and cellular uptake of various substances, which may be referred to herein as plasmaporation. Applicants also filed U.S. Provisional Application Ser. No. 61/911536 filed on Dec. 4, 2013 and U.S. Non-Provisional application Ser. No. 14/560,343 filed on Dec. 4, 2014, both of which are entitled Transdermal Delivery of DNA Vaccines Using Non-Thermal Plasma, and are both incorporated herein by reference in their entirety.

Plasmaporation uses non-thermal (cold) plasma, the fourth state of matter, for transdermal delivery of molecules, drugs, vaccines and the like through tissue and into cells. Non-thermal plasma is a partially ionized gas generated at atmospheric pressure using electricity. It is generated by the breakdown of air or other gases present between two electrodes under the application of sufficiently high voltage. A pulsed electric field used to generate the plasma opens up temporary pores in the skin and within cells to promote transdermal delivery and cellular uptake of molecules (including macromolecules), drugs, vaccines and the like. In some embodiments, for example, the temporary pores remain open for about 1 to about 5 minutes.

The electrode(s) generating the plasma are not in contact with the skin, no needles are required, and generation of non-thermal plasma directly on skin is rapid and painless. In exemplary embodiments with configurations where the electrodes are insulated, non-thermal plasma is formed by dielectric barrier discharge (DBD), which is safe and painless when applied to skin. The devices and techniques described herein result in more efficient and rapid means of plasmaporation skin treatments in a painless manner without the need for injection. Accordingly, the plasmaporation devices and techniques described herein can promote efficient intercellular delivery and intracellular uptake of various substances, including, for example, molecules, drugs, vaccines, and the like.

Plasmaporation has a number of other practical applications. In some embodiments, plasmaporation may be used to increase permeation of sanitizers, antimicrobials, surgical scrubs, and the like. Plasmaporation may be also be used to treat acne. First, plasmaporation may open the existing clogged pores as well as surrounding pores and sterilize the infected area. Second, plasmaporation allows antimicrobials and other acne medication to enter the pores. Plasmaporation may be also used to open pores and drive cosmetic related materials, such as, for example, collagen, BOTOX or other fillers into the skin to reduce wrinkles. Plasmaporation may be used to increase the absorption rate of moisturizers and thereby minimizes the “tack” associated with moisturizers that have not been fully absorbed.

In some embodiments, the skin may be preconditioned to temporarily alter the skin pH, moisture level, temperature, electrolyte concentration or the like. Preconditioning helps maximize speed and depth of permeation of active ingredients through pore formation without harming the skin.

In some embodiments, plasmaporation may be used in combination with low levels of non-irritating chemical skin permeation enhancers to achieve synergistic permeation of actives, including antimicrobials, cosmetic ingredients, vaccines, or drugs. Examples of chemical enhancers include dimethyl sulfoxide, azone, pyrrolidones, oxazolidinones, urea, oleic acid, ethanol, liposomes.

In some exemplary embodiments, plasmaporation involves the use of a planar DBD or a DBD jet plasma generator for needle-free transdermal delivery of macromolecules. Depending on the plasma dose, the depth of penetration of the macromolecules can be regulated to ensure delivery to a certain region of the skin, for example, a target layer such as the stratum corneum, epidermis, and/or dermis.

Applicants have demonstrated that plasmaporation can enhance transdermal delivery of topically applied dextran molecules with molecular weights up to 70 kDa across ex vivo porcine skin within 15 minutes and without creating skin damage, as described in the patent applications entitled Method and Apparatus for Delivery of Molecules Across Layers of the Skin on Sep. 27, 2013 and Sep. 29, 2013 incorporated herein. Others devices and techniques are described in U.S. Non-Provisional application Ser. No. 14/564,717 filed on Dec. 9, 2014, U.S. Non-Provisional application Ser. No. 14/610,467 filed on Jan. 30, 2015, and U.S. Non-Provisional application Ser. No. 14/967,512 filed on Dec. 14, 2015. All of these applications are incorporated by reference herein in their entirety.

The exemplary embodiments of apparatuses and methods disclosed herein use non-thermal plasmas to enable transdermal delivery of macromolecules, drugs, vaccines and the like, through the surface and into skin without harming the skin. Non-thermal plasma enabled skin poration provides a non-invasive, safe means for transdermal delivery and intracellular uptake of molecules, drugs and vaccines at room temperature and atmospheric pressure without the possible pain and other adverse side effects associated with electroporation. An additional benefit of using non-thermal plasma is that the generated reactive species sterilizes the skin during plasmaporation.

In the plasma phase, neutral gas atoms (or molecules), electrons, positive/negative ions, and radicals are generated. Their generation and concentration depend, in part, on the physical and chemical properties of the gas being used to generate the plasma as well as the electrical parameters used to generate the plasma. The strength of the electric field generated by non-thermal plasma on skin can be tuned in various ways, including, for example, by varying the time of plasma treatment; by varying the gap between the electrode and the skin; by varying the applied voltage; by varying the pulse duration; by varying the frequency; by varying the duty cycle; by adding conductive elements in contact with the skin; by varying the configurations and shapes of the conductive elements; by varying the placement of the conductive elements; by adding circuitry in operative communication with the conductive elements; by adjusting variable features or components of the circuitry; etc. These parameters can allow for the control of the depth and delivery amount of various substances associated with skin treatments, including, for example, macromolecules, drugs, vaccines and the like. Tuning and controlling the depth and distribution of the electric field generated during plasma-treatment of skin can allow the skin treatment to be directed to a targeted region of the skin, for example, a particular skin layer or layers, with an optimal dose and/or delivery process.

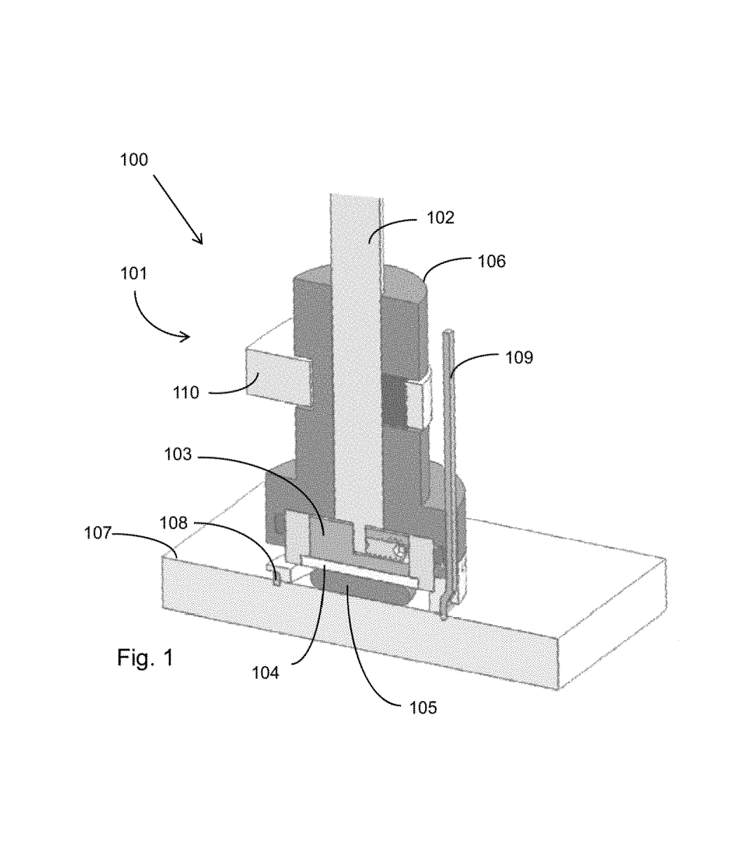

FIG. 1 illustrates a cross section of an exemplary skin treatment apparatus that includes an exemplary transdermal delivery system 100. Transdermal delivery system 100 includes a plasma generator 101. Plasma generator 101 includes a high voltage cable 102 connected to an electrode 103 on a first end and a high voltage power supply (not shown) on the second end. The power supply can be a pulsed DC, AC, pulsed AC, RF, microwave, or any other suitable power supply. The power supply may utilize one or more different wave forms, such as, for example, a constant, ramp-up, ramp-down, pulsed, nanosecond pulsed, microsecond pulsed, square, sinusoidal, random, in-phase, out-of-phase, and the like. In exemplary embodiments, the power supply generates microsecond (1-10 μs) and nanosecond (1-500 ns) duration pulses. In exemplary embodiments, the applied voltage can range from about 3 kV to about 30 kV with an operating frequency range from about 10 Hz to about 30 kHz. In exemplary embodiments, the power supply can operate in a continuous mode (e.g., for about 1-120 seconds) or in a pulsed mode (e.g., for about 1-100,000 pulses). In one embodiment, the pulses can be triggered manually, for example, for about 1 to about 200 pulses. In another embodiment, the pulses can be triggered automatically, for example, for about 1 to about 100,000 pulses. In any of these embodiments, the pulse duration and pulse interval may be separately specified or controlled. In various embodiments, the skin treatment time can range from a single pulse (e.g., about 10 ns) to a few minutes (e.g., about 120 s). In other embodiments, the pulses comprise a duty cycle from about 10% to about 100%. In other embodiments, the power supply can be battery-driven, integrated into the plasma generator 101 with the high voltage electrode 103, and/or part of an application, detection, and quantification module.

In this exemplary embodiment, plasma generator 101 is a non-thermal dielectric barrier discharge (DBD) generator. A dielectric barrier 104, for example, a quartz dielectric, is located below the high voltage electrode 103. Plasma 105 can be generated using this type of plasma generator, for example, by applying an alternating polarity pulsed voltage with nanosecond duration pulses. In one embodiment, the applied voltage may have a pulse width of between about 40-500 ns (single pulse to 20 kHz) with a rise time of 0.5-1 kV/ns and a magnitude of about 20 kV (peak-to-peak) at a power density of 0.01-100 W/cm². In one embodiment, a 1 mm thick clear quartz slide can be used as the insulating dielectric barrier 104 that covers the electrode 103. An exemplary electrode 103 includes about a 2.5 cm diameter copper electrode. In one embodiment, the discharge gap between the dielectric barrier 104 and the skin 107 is about 4 mm±1 mm. The high voltage electrode 103 and dielectric barrier 104 can be located within a housing 106, along with additional components.

Plasma 105 is generated by the plasma generator 101 in the air gap above skin 107 and can be in direct contact with the skin 107. In this embodiment, the plasma generator 101 also incorporates an exemplary conductive element 108 that is also in contact with the skin 107. In this embodiment, the conductive element 108 surrounds the generated plasma 105 and is supported by a plastic ring. Also in this embodiment, an optional connection post 109 is shown to facilitate circuit communication with the conductive element 108. Circuitry (not shown, described below) associated with the conductive element 108 may be placed in circuit communication with the conductive element 108 via the connection post 109. In one embodiment, the conductive element 108 or connection post 109 may be connected to ground, effectively grounding the conductive element 108. Grounding the conductive element 108 can include maintaining the conductive element 108 at ground potential and/or placing the conductive element 108 in circuit communication with earth ground. In another embodiment, a floating ground may be utilized. In this manner, the conductive element 108 can act as an electrode affecting the electric field associated with the plasma 105.

The plasma 105 and conductive element 108 direct the electric field associated with the plasma 105 through at least a region of the skin 107, depositing electrical charges that can develop a voltage potential across the skin, which leads to intracellular and intercellular poration. This plasmaporation is non-invasive since the plasma/high voltage electrode 103 is not in contact with the skin 107 being treated.

With respect to intracellular poration, the transmembrane voltage of fluid lipid bilayer membranes reaches at least about 0.2 V. The transmembrane voltage charges the lipid bilayer membranes, causes rapid, localized structural rearrangements within the membrane and causes transitions to water-filled membrane structures, which perforate the membrane forming “aqueous pathways” or “pores.” The aqueous pathways or pores allow an overall increase in ionic and molecular transport. The transmembrane voltage is believed to create primary membrane “pores” with a minimum radius of about approximately 1 nm. In addition, the applied electric field results in rapid polarization changes that deform mechanically unconstrained cell membranes (e.g., suspended vesicles and cells) and cause ionic charge redistribution governed by electrolyte conductivities.

The electrical pulses used to generate the plasma 105 also cause intercellular poration. The SC, which is about 15-25 μm thick, is the most electrically resistive part of skin. The application of electrical pulses used to generate the plasma 105 can give rise to a transdermal voltage ranging between about 50V and about 100V, which can cause poration of the multilamellar bilayers within the SC. At these levels of applied transdermal voltage, poration of cell linings of sweat ducts and hair follicles could also occur.

Upon stoppage of the plasma or removal of the plasma generator 101 from the treated area, the pores of the skin 107 tend to close again and thus, the process is reversible. Some pores may remain open for an extended period of time, during which molecules of a treatment substance can continue to cross the cell membrane via diffusion. It has been discovered that in some embodiments, the pores of the skin 107 remain open for less than about 5 minutes. Experimental results demonstrated that a 10 kDa Dextran molecule applied to a plasma treated area was transported through open pores in the SC when applied within 0 to about 5 minutes. In this embodiment, after 5 minutes, the 10 kDa Dextran molecules no longer passed through the SC. Experimental results also demonstrated that smaller molecules like nicotine or caffeine were transported through open pores in the SC when applied within 0-20 minutes. In this embodiment, after 20 minutes, the small molecules no longer passed through the SC.

When electric pulses are applied to the skin, the absorbed energy can cause localized heating and damage to the skin. Energy greater than 50 J/cm² deposited on intact skin results in second degree burns and thermal damage to the underlying intact skin. One method of overcoming this problem is to apply short duration pulses repetitively, which allows the same amount of energy that would otherwise cause damage to be transferred without causing localized heating and skin damage. In some embodiments, the energy deposited on intact skin is less than about 25 J/cm², in some embodiments, the energy deposited on intact skin is less than about 10 J/cm², in some embodiments, the energy deposited on intact skin is less than about 5 J/cm², and in some embodiments, the energy deposited on intact skin is less than about 3 J/cm². However, when treating wounds, the energy may be increased to, for example, 500 J/cm², without causing burns. In some embodiments, energy in the range of 500 J/cm² may be used to coagulate blood.

In addition, damage to the skin may occur from localized plasma micro-discharges, also known as “streamers,” that occur with non-uniform electric fields. This problem may be overcome by creating a uniform electric field, including, for example, by incorporation of the conductive element 108. Also, skin damage can be avoided by reducing the power level, frequency, duty-cycle and pulse duration of the power supply and by increasing the spacing of the air gap between the high voltage electrode 103 and the skin 107 to be treated. In this embodiment, the apparatus 100 also includes a strain relief locking mechanism 110 that may be incorporated into the housing 106 to lock the high voltage cable 102 into place. In another embodiment, the apparatus 100 may also includes an adjustable height feature (not shown) that may be incorporated into the housing 106 to lock a slidable shaft into position. The adjustable height feature can control the spacing between the plasma electrode 103 and the skin 107.

Typically, plasma is applied directly to the skin being treated, which means the electric field is directed only in one direction, which is directly into the skin. In this manner, it is difficult to control the distribution of the electric field in the skin. By placing a conductive element on the skin, such as, for example, the conductive element 108 shown in FIG. 1 above, the electric field in the skin 107 can be controlled spatially in three dimensions and also the strength of the electric field can be focused to be stronger in certain regions of the skin 107, such as, for example, typical treatment target areas of the skin: the SC and epidermis. In one embodiment, the conductive element 108 can be grounded or connected to ground via a resistive path. The electric field can also be controlled temporally, for example, by connecting the conductive element 108 to circuitry that includes a capacitive and/or inductive path. Various types of exemplary circuitry will be described in more detail below.

In various embodiments, the conductive element can be in many different forms (e.g. solid, liquid, gel, etc.), shapes (e.g., ring, straight line, rectangle, etc.), and geometries (e.g., segment, foam, patch, spacer, etc.). In different embodiments, the conductive element can be placed on the skin 107 or it can be a part of the plasma generator 101 (as shown in FIG. 1). Various types of conductive elements will be described in more detail below.

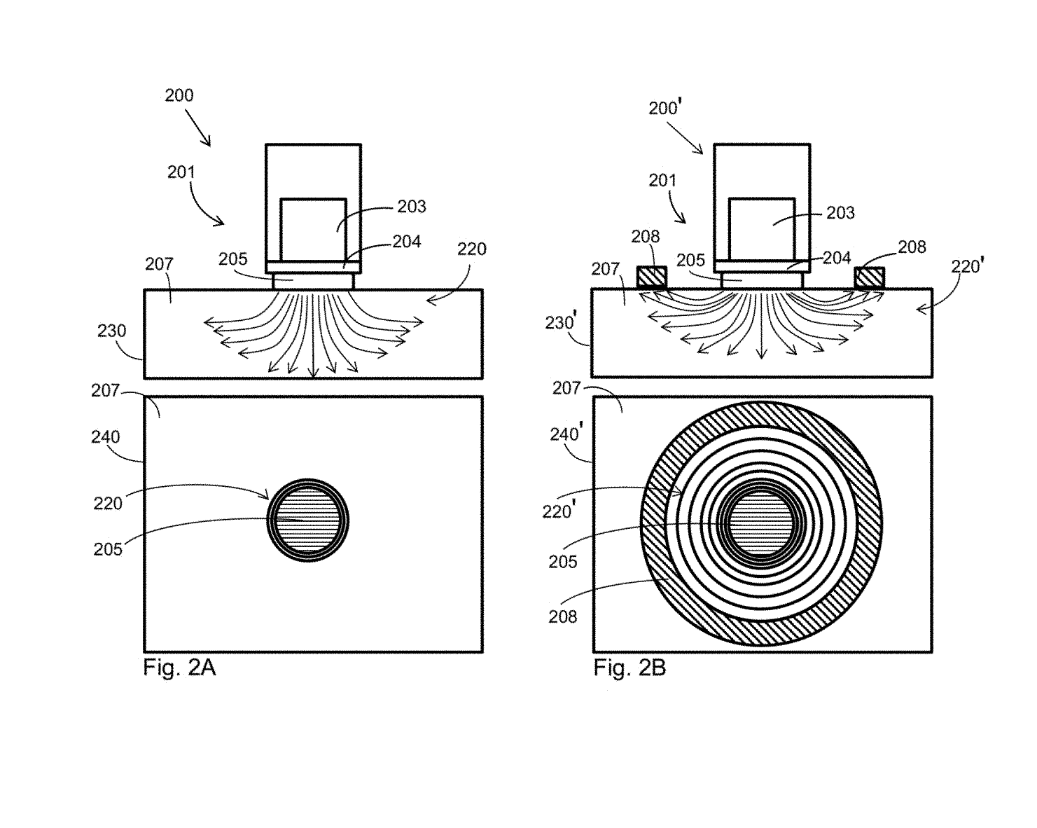

FIG. 2A illustrates a cross-section view of an exemplary skin treatment apparatus that includes an exemplary transdermal delivery system 200 and depictions of the electric field associated with the generated plasma without a conductive element. System 200 includes a plasma generator 201. Plasma generator 201 includes an electrode 203 and a dielectric barrier 204. Plasma 205 is generated by the plasma generator 201 above skin 207. Plasma 205 creates electric field 220 in the skin 207. Block 230 shows a cross-section of the spatial penetration and distribution of the electric field 220 in the skin 207. Block 240 is a plan view depicting the strength of the electric field 220 at the top layer of the skin 207. Blocks 230 and 240 show that the electric field 220 is strong only directly below and near the plasma 205 and with relatively deep penetration into the skin 207.

FIG. 2B illustrates a cross-section view of an exemplary skin treatment apparatus that includes an exemplary transdermal delivery system 200′ and depictions of the electric field associated with the generated plasma with a grounded conductive element. System 200′ includes the same plasma generator 201, electrode 203, and dielectric barrier 204 as system 200. However, in system 200′, grounded conductive element 208 is placed on the skin 207. The plasma 205 and conductive element 208 create electric field 220′ in the skin 207. Block 230′ shows a cross-section of the spatial penetration and distribution of the electric field 220′ in the skin 207. Block 240′ is a plan view depicting the strength of the electric field 220′ at the top layer of the skin 207. Blocks 230′ and 240′ show that the electric field 220′ is stronger throughout the skin 207 from below the plasma 205 to below the conductive element 208 and with shallower penetration into the skin 207.

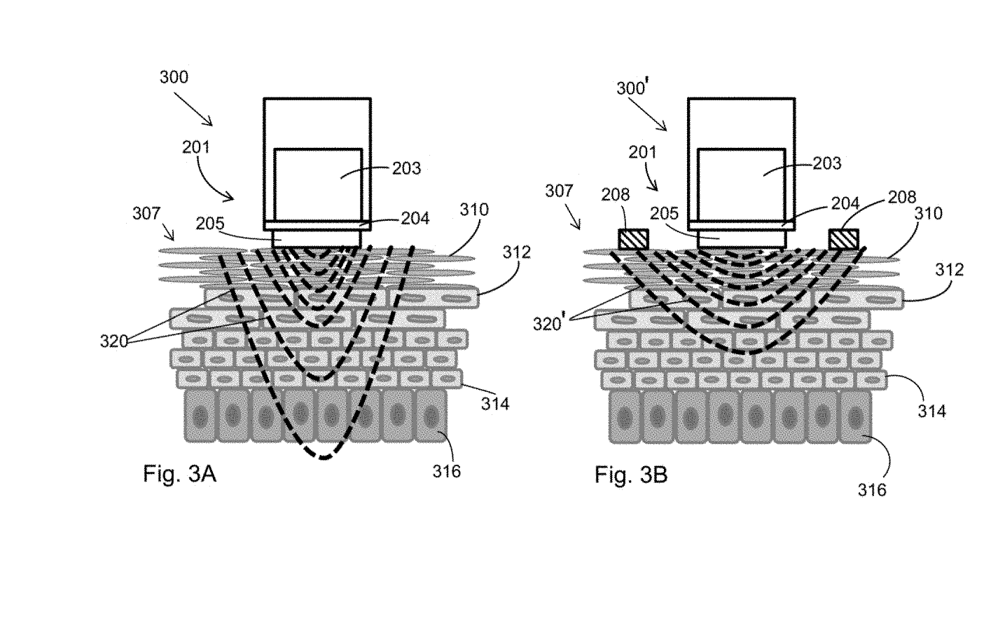

FIGS. 3A and 3B illustrate exemplary transdermal delivery systems 300 and 300′ (without and with conductive element 208, respectively) depicting the associated respective electric fields within a more detailed cross-section view of skin 307. Skin 307 is shown with various portions (layers) associated with intercellular and intracellular poration. These regions of skin 307 include the stratum corneum 310, viable epidermis 312, dermis 314, and subcutaneous tissue 316. In these figures, field lines 320, 320′ depict the density of the electric field throughout the skin 307 without and with grounded conductive element 208 directing the electric field, respectively.

Thus, as illustrated in FIGS. 2A and 3A, in the absence of conductive element 208 on the skin 207, 307, the electric field 220, 320 generated by the application of plasma 205 on the skin 207, 307 has a higher depth of permeation and a narrower area of distribution in the skin 207, 307, and the magnitude of the electric field 220, 320 is weaker in the SC 310 and epidermis 312. As illustrated in FIGS. 2B and 3B, when a grounded conductive element 208 (e.g., with a ring shape surrounding the plasma 205) is applied to the skin 207, 307, the electric field 220′, 320′ has a shallower depth of penetration and a wider distribution in the skin 207, 307. Additionally, the magnitude of electric field 220′, 320′ is significantly stronger in the SC 310 and epidermis 312, which is a typical target area for poration skin treatments, including, for example, to enhance transdermal drug delivery and intracellular uptake of drugs and molecules.

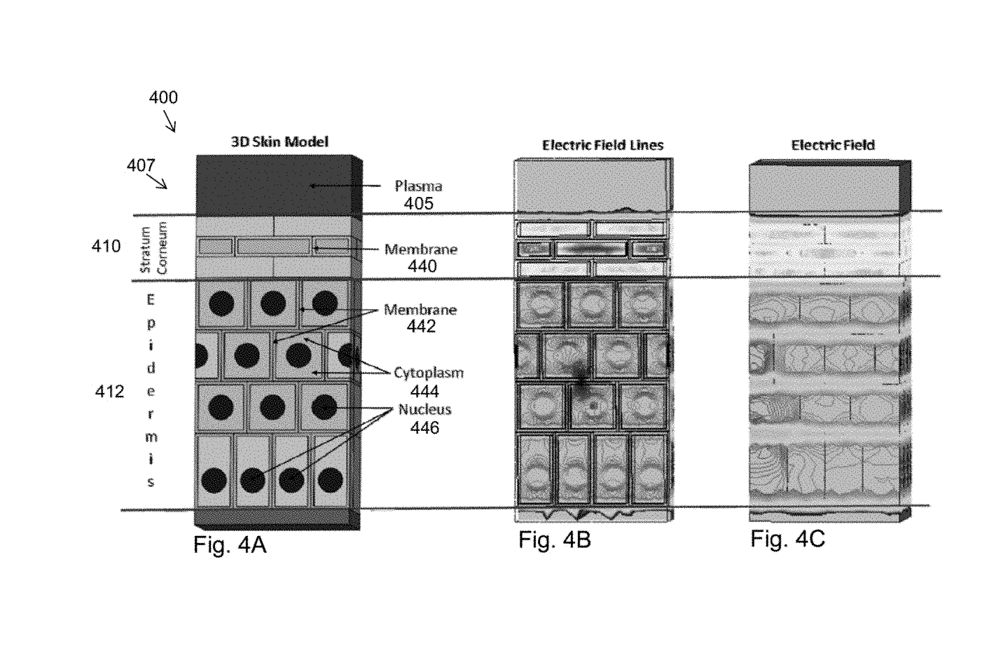

FIG. 4 is an illustration of a cross-section of model skin cells exposed to an exemplary electric field directed to the SC and epidermis layers of the skin. FIG. 4A depicts cells of skin model 400 where skin 407 is exposed to a plasma 405. Skin 407 is shown with a cross-section of targeted cells in the SC 410 and epidermis 412 layers. The cells in the SC 410 are shown with membranes 440. The cells in the epidermis 412 are shown with membranes 442, cytoplasm 444, and nuclei 446. FIGS. 4B and 4C show the field lines and electric field, respectively, within the skin cells shown in FIG. 4A, as the electric field associated with the plasma 405 is directed through these targeted skin cells in the SC 410 and epidermis 412 layers.

By using the conductive elements, the electric field associated with the plasma can be controlled and directed through the targeted regions of the skin. Localizing the effect of the plasma and its associated electric field allows users to target problem areas. Spatial control of the depth and width of the electric field can increase the magnitude of the electric field in the targeted areas and decrease the magnitude of the electric field in the non-targeted areas. Circuitry associated with the conductive elements can be utilized to ground the conductive elements, apply a bias voltage to the conductive elements, and/or introduce any number of resistive, capacitive, and inductive characteristics to the system. This circuitry adds the capability to modulate and tune the characteristics of the electric field for any type of skin treatment and application. Furthermore, this circuitry introduces temporal and dynamic control during and after plasma treatment.

Patient and user safety may also be improved due to the use of a conductive element (e.g., grounded) and a reduction in the current necessary to achieve the desired energy levels in the targeted areas. As discussed in detail below, the system can include a safety device, such as, for example, a position sensor, to ensure that the conductive element is in contact with the skin before plasma treatment begins. The system can also include a disposable contact surface associated with the conductive elements, such as, for example, a patch, to eliminate cross-contamination from patient to patient.

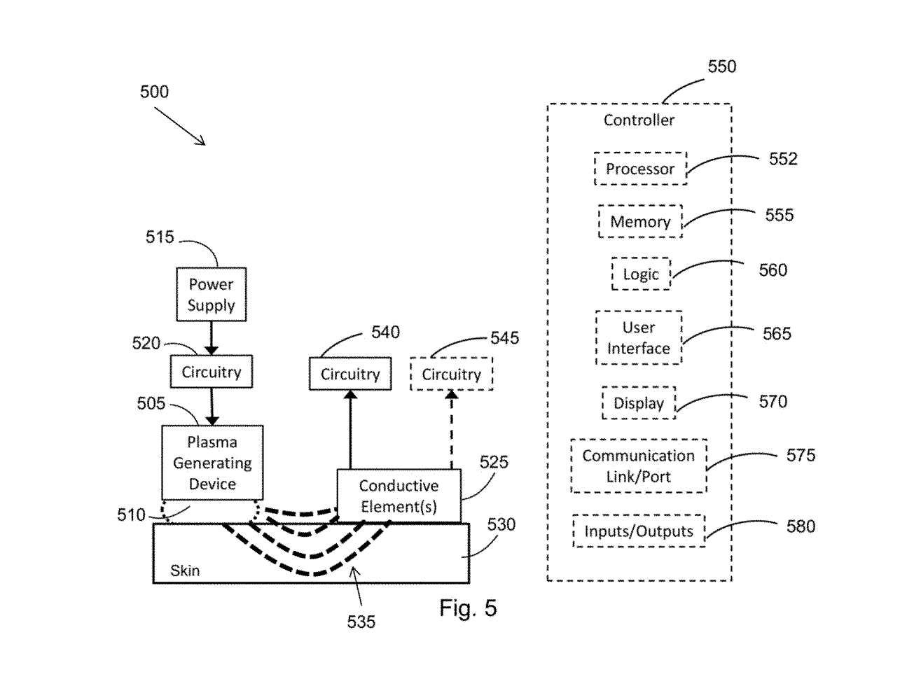

FIG. 5 is block diagram of an exemplary embodiment of a skin treatment apparatus 500 for controlling an electric field associated with a skin treatment. In this embodiment, the treatment apparatus 500 includes a plasma generating device 505 for generating a plasma 510, a power supply 515 for powering the plasma generating device 505, circuitry 520 for providing one or more electrical pulses to the plasma generating device 505, one or more conductive elements 525 in contact with the skin 530 for affecting an electric field 535 associated with the generated plasma 510, where at least a portion of the electric field 535 is directed through a region of the skin 530 by the plasma 510 and the conductive element 525. In some embodiments, the power supply 515 is battery-powered.

In various embodiments, the conductive element is in circuit communication with circuitry 540. In one embodiment, circuitry 540 places the conductive element 525 in circuit communication with ground. In another embodiment, conductive element 525 acts as a floating ground. Circuitry 540 can include the various grounding, resistive, capacitive, and inductive paths and features mentioned above and discussed in more detail below. These features allow the electric field 535 to be further controlled and/or tuned based on any particular application. In yet other embodiments, the apparatus 500 further includes circuitry 545, where circuitry 540 and circuitry 545 are each connected to different conductive elements 525 or subsets thereof, as described below in more detail. Circuitry 540 and 545 can have the same capabilities and features.

In other embodiments, the apparatus 500 may include an exemplary controller 550 for controlling one or more aspects of the apparatus 500. Controller 550 may include a processor 552, a memory 555, logic 560, user interface 565, display 570, communication link/port 575, inputs/outputs 580, and/or any other feature associated with a controller. In one embodiment, the controller 550 may be embodied as one or more computing devices, such as, for example, a computer (e.g., desktop, laptop, tablet), a portable smart device (e.g., smart phone, programmer, portable controller), etc. In some embodiments, the controller 550 may be associated with or include an application, detection, and/or quantification module.

Processor 552 may include device or combination of devices that function as a processor, as defined above, associated with the skin treatment apparatus or process. Logic 560 may include software for controlling and/or executing the skin treatment process, tuning routines, process sequences, safety checks, adjustments, etc. The memory 555 may store the logic 560, various algorithms associated with the logic 560, various settings for the power supply 515, plasma generating device 505, circuitry 520, 540, 545, etc. The memory 555 may be of any type or configuration, including, for example, local, remote, permanent, removable, centralized, shared, etc.

The memory 555 may also store an application database of treatment settings. For example, as apparatus 500 settings are tuned and/or optimized for various applications, the various application details and associated tuned parameters/settings of the apparatus 500 can be stored. Exemplary application details may include, for example, the treatment substance (e.g., drug), dosage, skin type, patient tolerance, etc. Exemplary tuned parameters/settings may include, for example, power supply 515 and associated circuitry 520 settings (e.g., type, voltage, polarity, waveform, frequency, pulse number and duration, duty cycle, etc.), plasma generating device 505 settings (plasma type, gas type, flow rate, etc.), spacing (height of plasma generating device 505 above skin), treatment times/routines, conductive element 525 features (e.g., placement, shape, size, thickness, material, segments, etc.), circuitry 540, 545 features (e.g., component (resistor, capacitor, inductor, etc.) values, component arrangement (e.g., series, parallel, etc.), component type (e.g., fixed, variable, etc.), grounding, etc.), etc.

The user interface 565 may include various input devices, such as, for example, buttons, dials, mouse, keyboard, touch-pad, etc. The display 570 may include one or more displays, including, for example, monitors, readouts, LCDs, LEDs, etc. The communication link/port 575 may include various devices suitable for any type of communication, including, for example, network connections (e.g., modem, LAN, WAN), wired (e.g., USB, Ethernet), wireless interfaces (e.g., Bluetooth, 802.11 standards, near field), portable storage medium interfaces (e.g., flash drive ports (e.g., memory sticks, USB, multimedia, SD, compact flash)), etc. Inputs/outputs 580 may include devices for receiving and/or transmitting various signals, information, readings, etc. associated with the apparatus 500, including to and/or from various devices, sensors, readouts, etc.

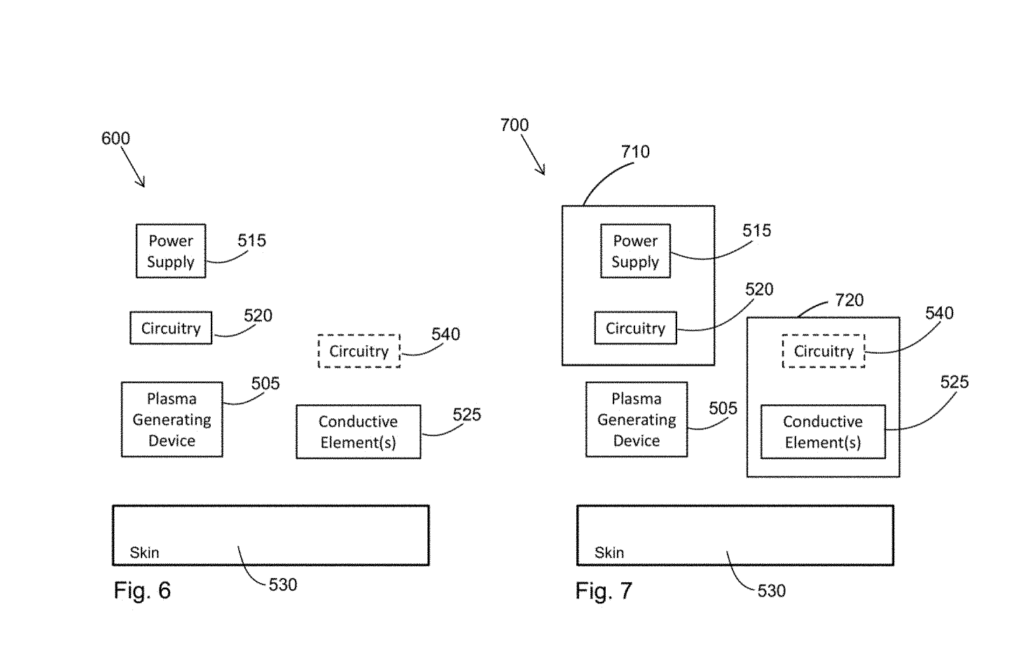

In various embodiments, the various components of apparatus 500 may be separate components in operative communication with each other or may be integrated to various degrees. The degree of integration may range from discrete components sharing a common housing to full integration into one or more integrated components or devices with combined capabilities. For example, as shown in FIG. 6, one embodiment of a skin treatment apparatus 600 includes the plasma generating device 505, the power supply 515, the circuitry 520, the conductive elements 525, and the circuitry 540 all as separate components. In another embodiment, as shown in FIG. 7, a skin treatment apparatus 700 includes a power device 710 that includes the power supply 515 and the circuitry 520 and a conductive assembly 720 that includes the conductive elements 525 and the circuitry 540.

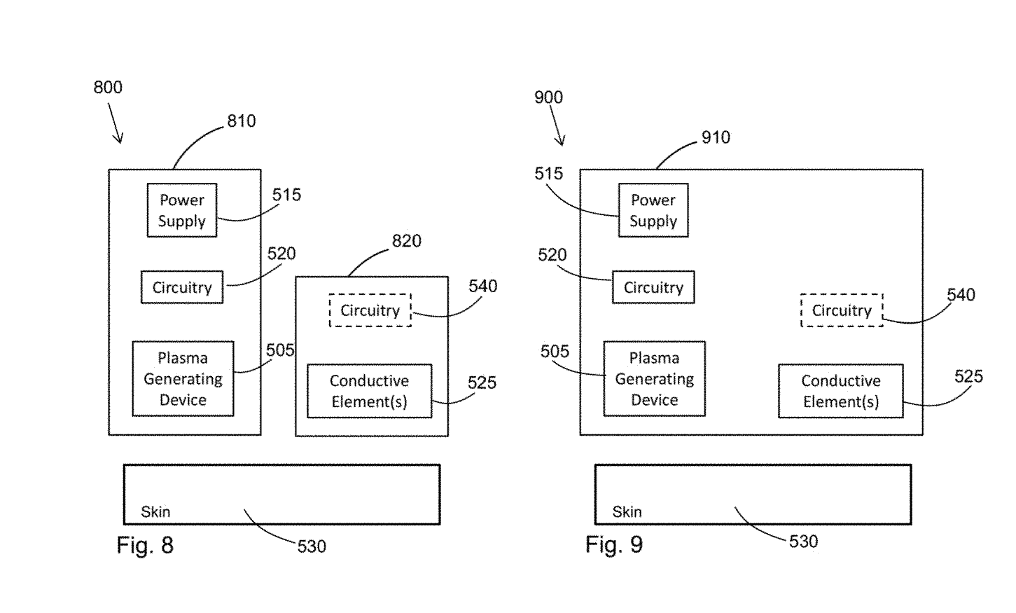

In another embodiment, as shown in FIG. 8, a skin treatment apparatus 800 includes a plasma generator 810 that includes the plasma generating device 505, the power supply 515, and the circuitry 520 and a conductive assembly 820 that includes the conductive elements 525 and the circuitry 540. In another embodiment, as shown in FIG. 9, a skin treatment apparatus 900 includes a plasma poration device 910 that integrates the plasma generating device 505, the power supply 515, the circuitry 520, the conductive elements 525, and the circuitry 540.

Although FIGS. 6-9 do not show the exemplary controller 550 and its associated components, any or all of these devices 550, 552, 555, 560, 565, 570, 575, 580 may be integrated with any of the components and devices shown in FIGS. 6-9 in other embodiments.

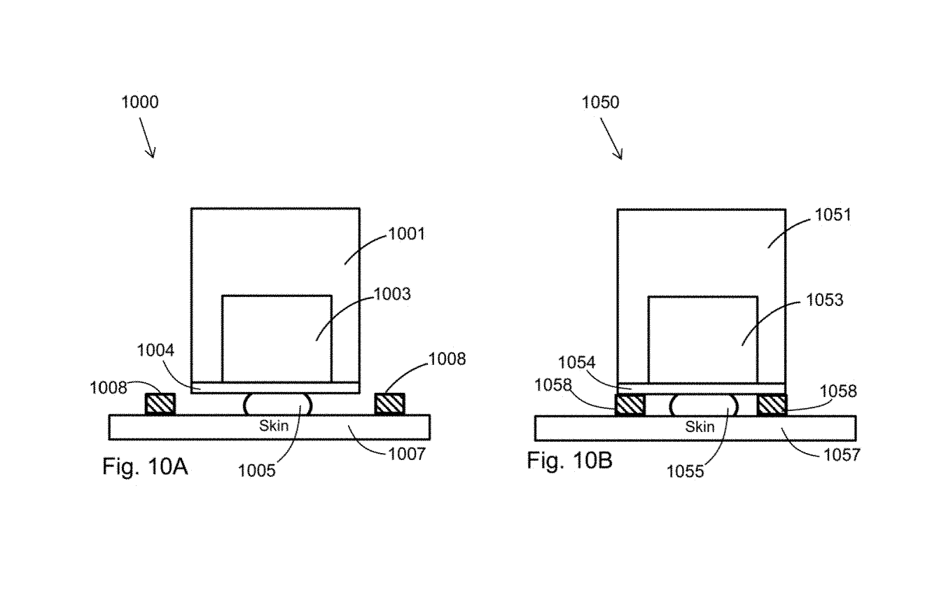

FIG. 10A illustrates a cross-section of an exemplary skin treatment system 1000 with a non-thermal DBD generator. System 1000 includes a plasma generator 1001 integrated with an electrode 1003 and a dielectric barrier 1004 that creates a plasma 1005 above skin 1007. In system 1000, a ring-shaped conductive element 1008 is placed on the skin 1007 in a manner that surrounds the plasma 1005 above the skin 1007. The plasma 1005 and conductive element 1008 create an electric field (not shown) in a region of the skin 1007. The plasma generator 1001 and the conductive element 1008 may be in circuit communication with associated circuitry and/or other components.

FIG. 10B illustrates a cross-section of another exemplary skin treatment system 1050 with a non-thermal DBD generator. System 1050 includes a plasma generator 1051 integrated with an electrode 1053 and a dielectric barrier 1054 that creates a plasma 1055 above skin 1057. Furthermore, in system 1050, a ring-shaped conductive element 1058 is also integrated with the plasma generator 1051 in a manner that surrounds the plasma 1055. In another embodiment, system 1050 may also include a height-adjustment mechanism (not shown) associated with the integrated conductive element 1058 such that the spacing between the plasma generator 1051 and the conductive element 1058 dictates the height of the plasma generator 1051 above the skin 1057. I.e., in this embodiment, when the conductive element 1058 comes in contact with the skin 1057, the plasma generator 1051 will be the correct height above the skin 1057. The plasma 1055 and the conductive element 1058 create an electric field (not shown) in a region of the skin 1057. The plasma generator 1051 and the conductive element 1058 may be in circuit communication with associated circuitry and/or other components.

FIG. 11A illustrates a cross-section of an exemplary skin treatment system 1100 with a floating-electrode DBD plasma generator that generates a plasma “jet.” System 1100 includes a plasma generator 1101 integrated with a tubular electrode 1103 and a dielectric barrier 1104 (e.g., a borosilicate glass tube) that generates a plasma “jet” 1105 above skin 1107. In system 1100, a ring-shaped conductive element 1108 is placed on the skin 1107 in a manner that surrounds the plasma 1105 above the skin 1107. The plasma 1105 and conductive element 1108 create an electric field (not shown) in a region of the skin 1107. The plasma generator 1101 and the conductive element 1108 may be in circuit communication with associated circuitry and/or other components.

FIG. 11B illustrates a cross-section of another exemplary skin treatment system 1150 with a floating-electrode DBD plasma generator that generates a plasma “jet.” System 1150 includes a plasma generator 1151 integrated with a tubular electrode 1153 and a dielectric barrier 1154 (e.g., a borosilicate glass tube) that generates a plasma “jet” 1155 above skin 1157. Furthermore, in system 1150, a ring-shaped conductive element 1158 is also integrated with the plasma generator 1151 in a manner that surrounds the plasma 1155. The plasma generator 1151 of system 1150 also has a conductive post 1160 that connects the conductive element 1158 to the plasma generator 1151. In another embodiment, system 1150 may also include a height-adjustment mechanism (not shown) associated with the integrated conductive element 1158 such that the spacing between the plasma generator 1151 and the conductive element 1158 dictates the height of the plasma generator 1151 above the skin 1157. The plasma 1155 and conductive element 1158 create an electric field (not shown) in a region of the skin 1157. The plasma generator 1151 and the conductive element 1158 may be in circuit communication with associated circuitry and/or other components.

Plasma generators 1101, 1151 utilize a gas feed. Exemplary gases that may be used to feed the plasma jet 1105, 1155 include air, He, He+O₂, N₂, He+N₂, Ar, Ar+O₂, Ar+N₂, and the like. Gases resulting from the evaporation of liquid solutions can also be used. Examples of vaporized liquids may include water, ethanol, organic solvents and the like. These vaporized liquids may be mixed with additive compounds. The evaporated liquids and additives may be used with the gases identified above in various concentrations or without the gases. During operation, the plasma jet 1105, 1155 is in direct contact with the skin 1107, 1157.

Other types of plasma generators may be used for transdermal delivery systems, such as, for example, nanosecond pulsed DBD plasma, microsecond pulsed DBD plasma, sinusoidal DBD plasma, corona discharge, glow discharge, resistive barrier discharge plasma, surface DBD plasma, 2-D or 3-D array of DBD plasma jets operating under a continuous mode or under a controlled duty cycle ranging from 1-100% and the like. It is important to note that not all plasma generators may be used to successfully induce poration. Thermal plasmas, gliding arc discharges, DC hollow cathode discharge, and plasmatron generators are examples of plasma generators that are not suitable for use in plasmaporation. Such plasma generators either deliver conduction current, which causes thermal damage, muscle contraction and pain or do not deliver sufficient charges to the substrate being treated, which would mean no or very weak applied electric field and hence no induced poration.

Suitable plasma generators have dominating currents that are displacement currents at low power and/or high frequencies. Displacement current has units of electric current density, and an associated magnetic field just as conduction current has, however, it is not an electric current of moving charges, but rather a time-varying electric field. The electric field is applied to the skin by an insulated high-voltage electrode that is not in contact with the skin. Because this electrode is insulated and is not in contact with the skin, there is no flow of conduction current into the skin, which would cause thermal damage, muscle contraction and pain that is associated with electroporation.

For larger treatment areas, electrode configurations consisting of multiple plasma jets or larger area flat electrodes may be used. In the case of more complex 3D surfaces, a controlled plasma module may move around a stationary target or the surface to be exposed to the plasma may be placed on a movable stage. In some embodiments, one or more plasma jets or can be attached to a robotic arm that is programmed to move in a manner that exposes one or more target areas to a plasma plume or jet.

In addition, in some embodiments, the plasma generator may be coupled with a biomolecule/drug delivery system, where molecules may be transported to the treatment area through needle-free injection, evaporation, spraying and or misting. In some embodiments, this may assist with the pretreatment of the surface.

In some embodiments where it is essential to reduce the plasma temperature and enhance skin permeation following plasmaporation it is beneficial to generate non-thermal plasma using He, Ar, Ne, Xe and the like, air, or mixtures of inert gases with small percentage (0.5%-20%) of other gases such as O₂ and N₂ and mixtures of inert gases with vaporized liquids including water, DMSO, ethanol, isopropyl alcohol, n-butanol, with or without additives and the like.

Referring back to FIGS. 10 and 11, the conductive element may be attached to the plasma generating device or placed on the skin before the skin treatment. In various embodiments, the conductive element may be permanent or temporary and reusable or disposable. An adhesive may be used to attach the conductive element to the plasma generating device or to the skin.

In other embodiments, the conductive element makes contact with the skin via another conductive device, such as, for example, a conductive patch. The conductive patch may be attached to the conductive element or placed on the skin before the skin treatment. The conductive patch may have a shape that matches the shape of the conductive element that contacts the skin. For example, if the conductive element is ring-shaped, the conductive patch may have a ring shape about the same size as or slightly larger than the conductive element. In one embodiment, the conductive patch is cleanable, temporary, and/or disposable. In this manner, the conductive patch may be the only component of the skin treatment apparatus or transdermal delivery system that makes contact with the patient during the skin treatment. An adhesive may be used to attach the conductive patch to the conductive element or to the skin. In some embodiments, the conductive patch comprises a releasable adhesive.

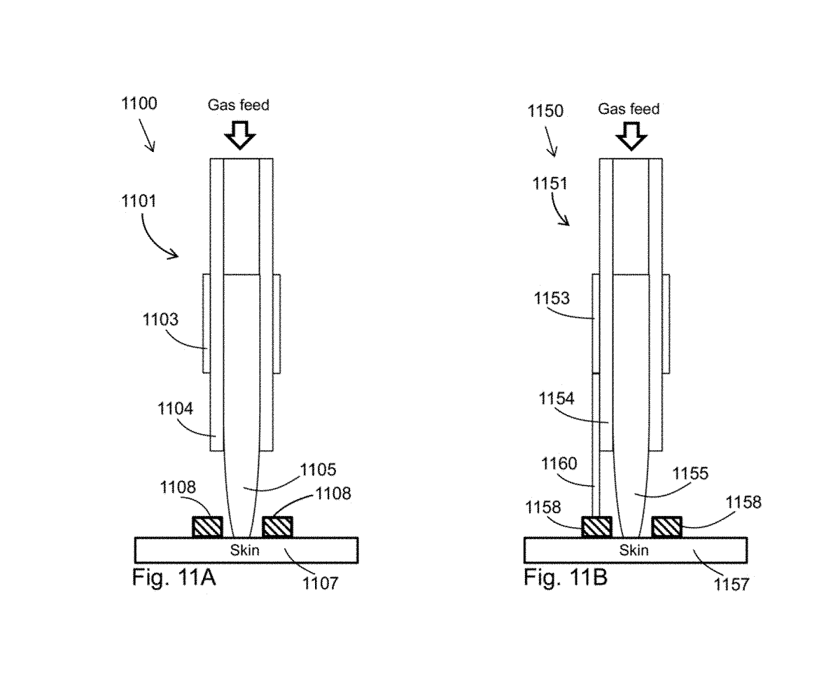

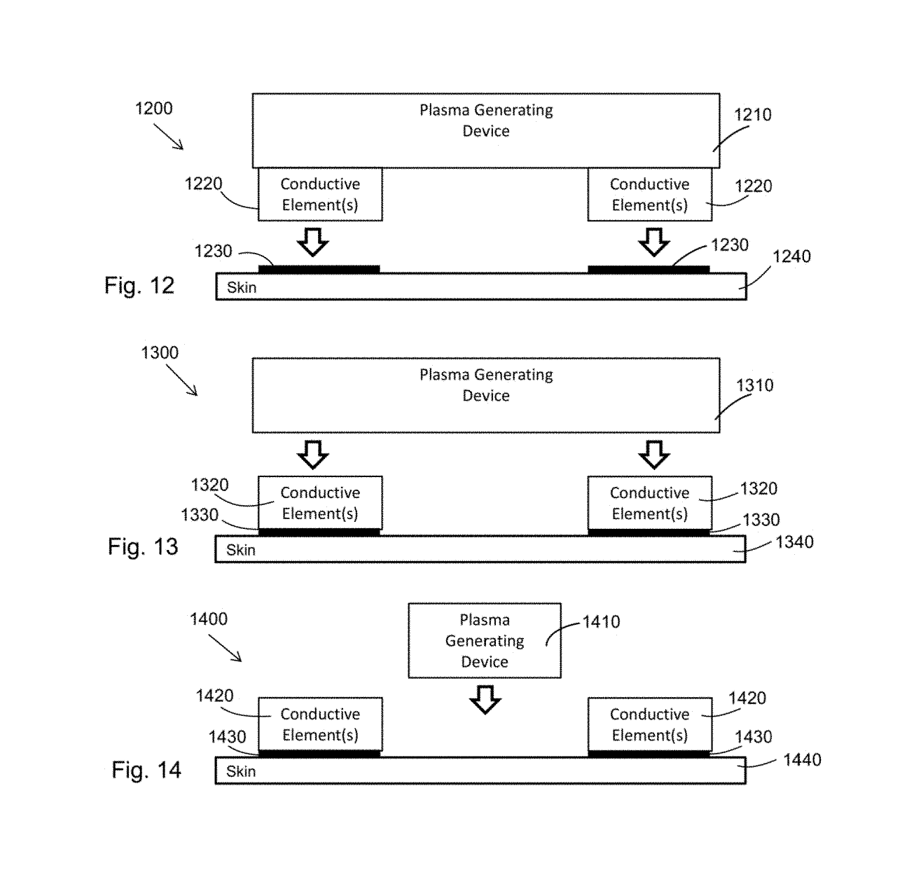

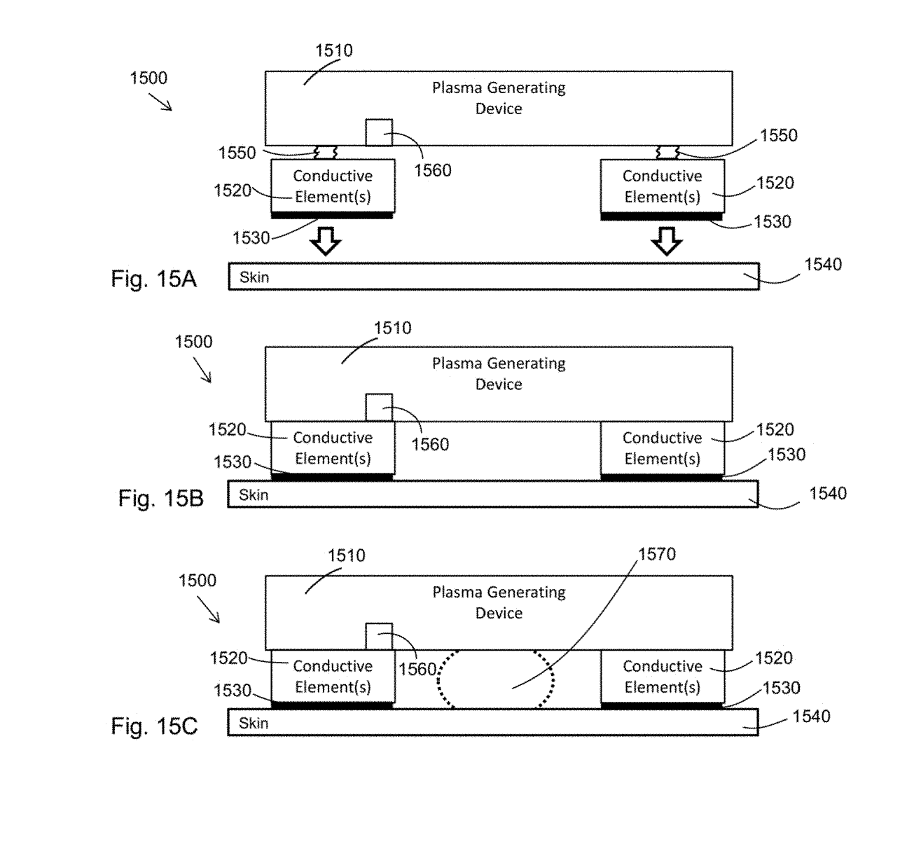

FIG. 12 illustrates a cross-section of an exemplary skin treatment system 1200 with a plasma generating device 1210 and a ring-shaped conductive element 1220. An exemplary ring-shaped conductive patch 1230 is shown attached to the skin 1240. In this embodiment, the conductive element 1220 is integrated with the plasma generating device 1210. Also in this embodiment, the conductive patch 1230 is attached to the skin 1240 before the conductive element 1220 makes contact with the skin 1240 via the conductive patch 1230. In another embodiment, the conductive patch 1230 may be attached to the conductive element 1220 before the conductive element 1220 makes contact with the skin 1240 via the conductive patch 1230. (See, e.g., the embodiment shown in FIG. 15 below.)

In this embodiment, during skin treatment, the plasma generating device 1210 with the integrated conductive element 1220 is placed into contact with the skin 1240 by aligning the footprint of the conductive element 1220 with the conductive patch 1230 already attached to the skin 1240. Once the conductive element 1220 is in contact with the conductive patch 1230, the plasma generating device 1210 may generate plasma.

FIG. 13 illustrates a cross-section of another exemplary skin treatment system 1300 with a plasma generating device 1310 and a ring-shaped conductive element 1320. An exemplary ring-shaped conductive patch 1330 is shown between the conductive element 1320 and the skin 1340. In this embodiment, the conductive element 1320 is not integrated with the plasma generating device 1310 but does need to make contact with the plasma generating device 1310, for example, to become in circuit communication with the plasma generating device 1310. Also in this embodiment, the conductive patch 1330 may be attached to the skin 1340 or the conductive element 1320 as long as it is located between the conductive element 1320 and the skin 1340. In this embodiment, during skin treatment, the plasma generating device 1310 is placed into contact with the conductive element 1320, which is already in contact with the skin 1340 via the conductive patch 1330, by aligning the footprint of the plasma generating device 1310 with the conductive element 1320. Once the plasma generating device 1310 is in contact with the conductive element 1320, the plasma generating device 1310 may generate plasma.

FIG. 14 illustrates a cross-section of another exemplary skin treatment system 1400 with a plasma generating device 1410 and a ring-shaped conductive element 1420. An exemplary ring-shaped conductive patch 1430 is shown between the conductive element 1420 and the skin 1440. In this embodiment, the conductive element 1420 is not integrated with the plasma generating device 1410 and does not need to make contact with the plasma generating device 1410 before the skin treatment. Also in this embodiment, the conductive patch 1430 may be attached to the skin 1440 or the conductive element 1420 as long as it is located between the conductive element 1420 and the skin 1440. In this embodiment, during skin treatment, the plasma generating device 1410 is not placed into contact with the conductive element 1420, which is in contact with the skin 1440 via the conductive patch 1430. Once the plasma generating device 1410 is in position relative to the conductive element 1420, the plasma generating device 1410 may generate plasma.

In any or all of these embodiments, the conductive element and/or the conductive patch may be configured for disposal after every use, after every patient, or according to some other regimen or protocol. In some embodiments, disposal includes discarding without any reuse. In other embodiments, the conductive element and/or the conductive patch may be reused, but configured for cleaning or sterilization after every use, after every patient, or according to some other regimen or protocol.

In some embodiments, a device may be used to sense and/or verify that the conductive element is in contact (circuit communication) with the skin before the plasma is generated. For example, the device may include a position sensor, a switch, a proximity sensor, or the like. This feature may be associated with a safety interlock system. The sensing device may be in circuit communication with a controller, such as, for example, controller 550 shown in FIG. 5, where the controller controls (e.g., enables and disables) the plasma generating device based on an input or signal from the position sensing device. In another embodiment, the sensing device is a continuity circuit from the conductive element to the skin, e.g., by checking the resistance to ground.

FIG. 15 illustrates a cross-section of an exemplary skin treatment system 1500 with a plasma generating device 1510, a ring-shaped conductive element 1520, and a ring-shaped conductive patch 1530 applied to the conductive element 1520. In this embodiment, the conductive element 1520 is integrated with the plasma generating device 1510 and is spring-loaded with one or more springs 1550. The spring 1550 biases the conductive element 1520 into a non-ready position. The position of the conductive element 1520 is sensed by a position-sensing device 1560.

As shown in FIG. 15A, the system 1500 is in a non-ready position since the conductive element 1520 is not in contact with the skin 1540. In this state, the position-sensing device 1560 recognizes the non-ready position of the conductive element 1520. For example, in one embodiment, the sensing device 1560 is a switch that is in an open or a closed condition when the conductive element 1520 is not in contact with the skin 1540. In FIG. 15B, the system 1500 is in a ready position since the conductive element 1520 is now in contact with the skin 1540 after compressing the spring 1550 of the spring-loaded mechanism. In this state, the position-sensing device 1560 recognizes the ready position of the conductive element 1520. For example, in one embodiment, the sensing device 1560 is a switch that changes state to a closed or an open condition, with respect to its state shown in FIG. 15A, when the conductive element 1520 is in contact with the skin 1540. In FIG. 15C, the system 1500 is in a ready position and the plasma generating device 1510 generates plasma 1570.

The conductive elements may be made of any conductive material (e.g., copper, aluminum, tungsten, silver, gold, titanium, palladium, conductive foam, conductive polymer, ITO, reticulated vitreous carbon, etc.). The conductive elements may be in many different forms (e.g. solid, liquid, gel, etc.). The conductive elements may be any shape suitable for an application, including planar shapes (i.e., the shape of the surface of the conductive element contacting the skin) and cross-sectional shapes. For example, the planar shape of the conductive element may be a straight line, a circular shape, an oval shape, a square shape, a rectangular shape, etc. The cross-sectional shape may be any shape suitable for providing sufficient contact with the skin.

In some embodiments, the top and bottom surfaces of the conductive element may be different. For example, the bottom surface may be configured or adapted to interface with the skin (e.g., for a good conductive surface-to-surface connection without connectors) and the top surface may be differently configured or adapted to interface with the plasma generating device (e.g., with an electrical connector, threaded connectors, etc.).

The conductive element may surround the plasma site or may be in close proximity to or adjacent to the plasma site. In some embodiments the conductive element includes a plurality of conductive elements. These elements may direct the electric field to different regions of the skin or may be used to more evenly distribute the electric field. In some embodiments, two or more conductive elements may form peripheries around the plasma site and may be arranged concentrically. In some embodiments the conductive element includes a plurality of segmented conductive elements. The segmented elements may exhibit the same features of non-segmented elements, including, for example, materials, overall shape, connections to other components, etc. In one embodiment, the segmented conductive elements are equally spaced and surround the plasma site.

The conductive element may have a size much larger than, slightly larger than, about the same size as the plasma treatment area. As discussed above, the conductive element may be secured to the skin (or any tissue) by having an adhesive backing or a conductive patch. In one embodiment, during storage, the adhesive backing can be sealed. During treatment, the adhesive backing can be exposed and placed in good contact with skin outside of or near the area to be plasma treated. Proper contact between the conductive element and the skin may be ensured before plasma treatment by a safety device as described above. The conductive element or an associated conductive patch may be integrated with, permanently attach to, or be a consumable item that temporarily attaches to the plasma generating device. A replaceable conductive element and/or conductive patch could eliminate cross contamination from one patient to another and also make cleaning components of the treatment apparatus easier between uses.

The conductive elements may be floating, grounded, connected to a bias voltage, and/or connected to circuitry. In embodiments with more than one conductive element or segmented conductive elements, subsets of the conductive elements may be connected differently in any combination.

In various embodiments, the circuitry connected to the conductive element can include resistors, capacitors, inductors, and combinations thereof. Circuitry associated with the conductive element can include any components and/or devices that achieve the resistive, capacitive, inductive and/or other characteristics desired for any particular application, including analog circuits, digital circuits, discrete components, integrated circuits, combinations thereof, etc. Any of these devices and/or components may have variable features or values, capable of adjustment. In some embodiments, various bias voltages can also be applied to the conductive element via the circuitry. Some or all of the circuitry may be discrete components, may be connected via a module or circuit, may be integrated with the plasma generating device, and/or may be integrated with the conductive element or a conductive assembly.

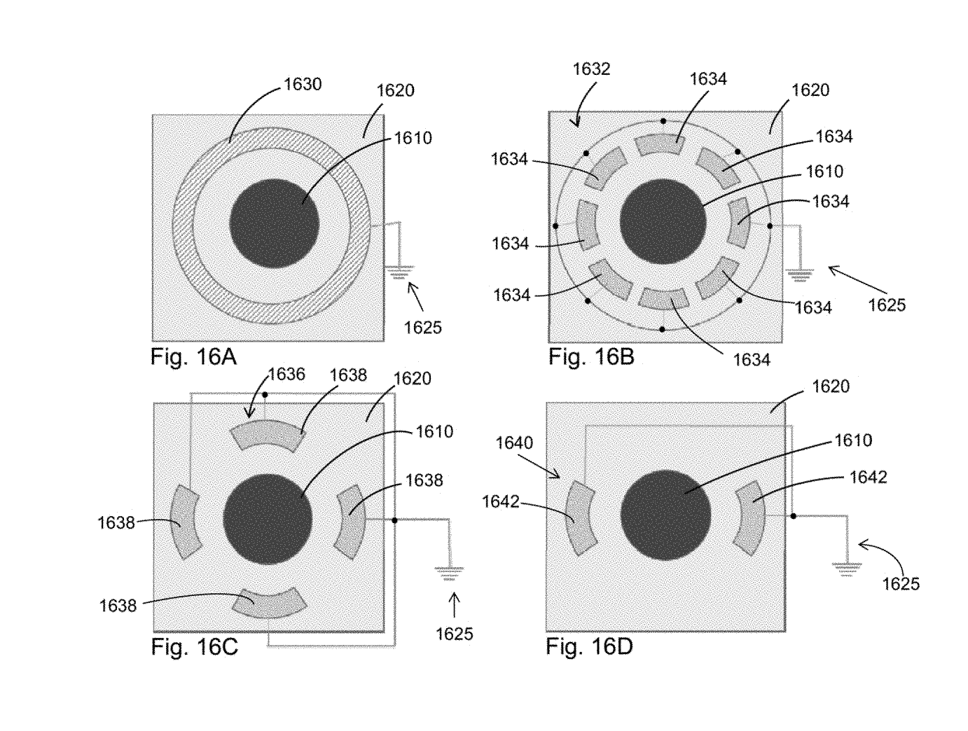

FIG. 16 shows various exemplary conductive elements connected to ground. In these embodiments, the conductive elements surround a plasma site 1610 on skin 1620 in a ring or circular planar shape. Circuitry 1625 places the conductive elements in circuit communication with ground. In particular: FIG. 16A shows an exemplary single conductive element 1630; FIG. 16B shows an exemplary segmented conductive element 1632 with eight evenly spaced and same-size segments 1634; FIG. 16C shows an exemplary segmented conductive element 1636 with four evenly spaced and same-size segments 1638; and FIG. 16D shows an exemplary segmented conductive element 1640 with two evenly spaced and same-size segments 1642. In all of these embodiments, all of the conductive elements are in circuit communication with ground. In other embodiments, the segments can be arranged in any number of configurations suitable for various applications, including, for example, with unevenly spaced segments, different-sized segments, different spacing between segments (e.g., more or less), different spacing between the segments and the plasma site 1610 (e.g., more or less), uneven numbers of segments, etc.

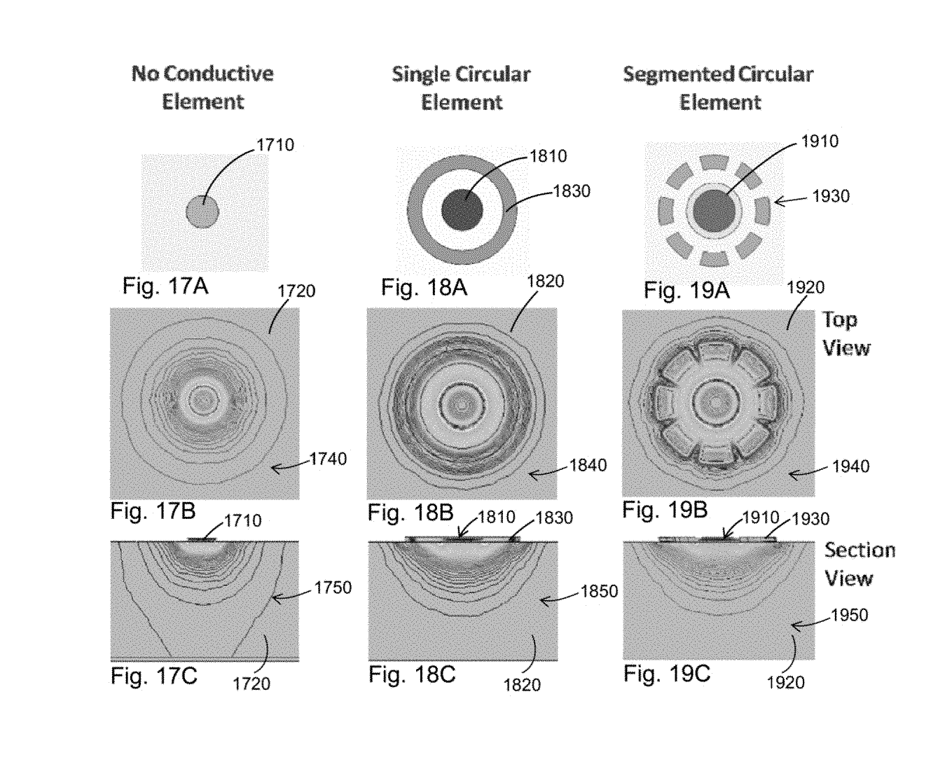

FIGS. 17-22 show various exemplary configurations of conductive elements and the associated electric fields created in conjunction with an exemplary plasma generator of a transdermal delivery system, such as, for example, one of those described above. The Top View is a plan view depicting the electric field at the top layer of the skin. The Section View shows a cross-section of the spatial penetration and distribution of the electric field in the skin.

In FIG. 17, a plasma 1710 is generated above skin 1720 without any conductive element(s). FIG. 17A shows the plasma 1710; FIG. 17B shows the electric field 1740 at the top of the skin 1720; and FIG. 17C shows a cross-section of the electric field 1750 throughout the skin 1720.

FIG. 18 shows a plasma 1810 generated above skin 1820 with a singular circular conductive element 1830 (e.g., conductive element 1630 from FIG. 16A) connected to ground (not shown). FIG. 18A shows the conductive element 1830 surrounding the plasma 1810; FIG. 18B shows the electric field 1840 at the top of the skin 1820; and FIG. 18C shows a cross-section of the electric field 1850 throughout the skin 1820.

FIG. 19 shows a plasma 1910 generated above skin 1920 with a segmented circular conductive element 1930 (e.g., conductive elements 1634 from FIG. 16B) connected to ground (not shown). FIG. 19A shows the conductive element 1930 surrounding the plasma 1910; FIG. 19B shows the electric field 1940 at the top of the skin 1920; and FIG. 19C shows a cross-section of the electric field 1950 throughout the skin 1920.

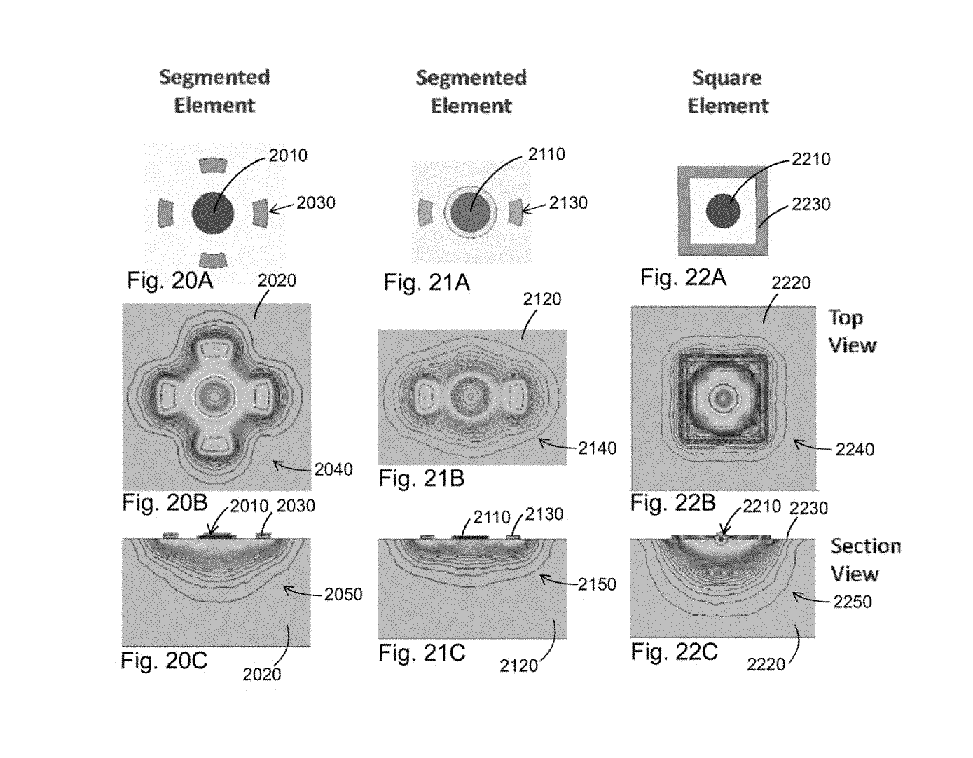

FIG. 20 shows a plasma 2010 generated above skin 2020 with a segmented circular conductive element 2030 (e.g., conductive elements 1638 from FIG. 16C) connected to ground (not shown). FIG. 20A shows the conductive element 2030 surrounding the plasma 2010; FIG. 20B shows the electric field 2040 at the top of the skin 2020; and FIG. 20C shows a cross-section of the electric field 2050 throughout the skin 2020.

FIG. 21 shows a plasma 2110 generated above skin 2120 with a segmented circular conductive element 2130 (e.g., conductive elements 1642 from FIG. 16D) connected to ground (not shown). FIG. 21A shows the conductive element 2130 surrounding the plasma 2110; FIG. 21B shows the electric field 2140 at the top of the skin 2120; and FIG. 21C shows a cross-section of the electric field 2150 throughout the skin 2120.

FIG. 22 shows a plasma 2210 generated above skin 2220 with a singular square-shaped conductive element 2230 connected to ground (not shown). FIG. 22A shows the conductive element 2230 surrounding the plasma 2210; FIG. 22B shows the electric field 2240 at the top of the skin 2220; and FIG. 22C shows a cross-section of the electric field 2250 throughout the skin 2220.

When compared to the electric fields associated with the embodiments with conductive elements, the electric field 1740, 1750 is stronger directly below and near the plasma 1710 and with relatively deep penetration into the skin 1720. Electric fields 1840, 1850, 1940, 1950, 2040, 2050, 2140, 2150, 2240, 2250 with conductive elements are generally stronger throughout the skin 1820, 1920, 2020, 2120, 2220 from below the plasma 1810, 1910, 2010, 2110, 2210 to below the conductive element 1830, 1930, 2030, 2130, 2230 and with shallower penetration into the skin 1820, 1920, 2020, 2120, 2220. However, the electric fields 1840, 1850, 1940, 1950, 2040, 2050, 2140, 2150, 2240, 2250 associated with the different configurations of conductive elements 1830, 1930, 2030, 2130, 2230 demonstrate that these respective electric fields can be spatially tuned and directed through different regions of the skin based on the configurations of conductive elements. In this manner, various conductive element configurations may be more suitable for different applications.

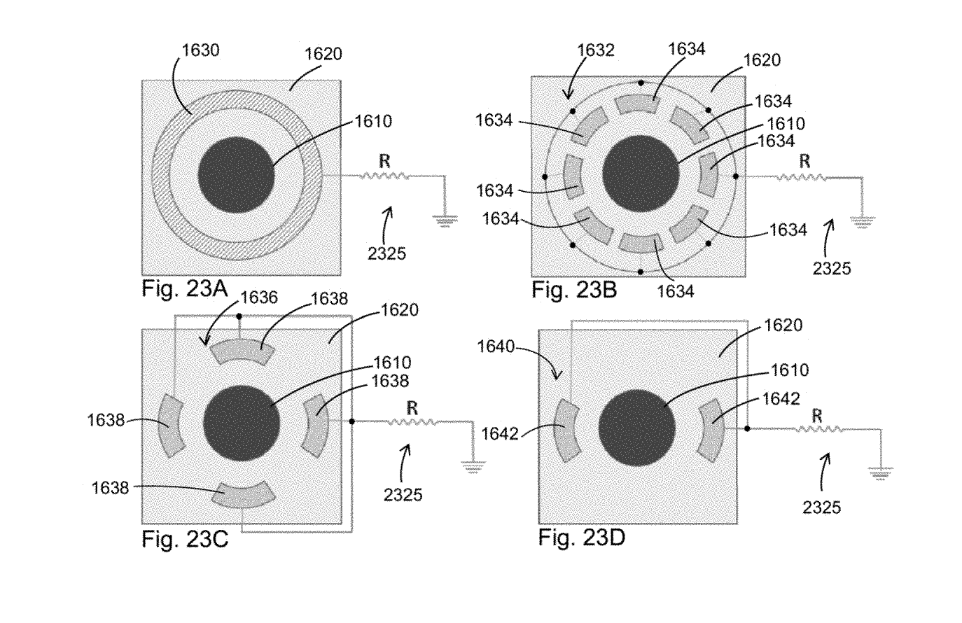

FIG. 23 shows various exemplary conductive elements connected to ground via a resistor R. In these embodiments, the conductive elements in FIGS. 23A-23D and their respective configurations mimic those shown in FIGS. 16A-6D, respectively, but with circuitry 2325 including resistor R to ground. Introducing resistor R between the conductive elements 1630, 1632, 1636, 1640 and the ground can limit the current flowing to ground during plasma application and enhance the electric field in the skin 1620. Additionally, the resistor R can provide a safety aspect to the patient and the user in the case of high current spikes. Circuitry 2325 with resistor R also allows further tuning of the electric field by controlling the duration of peak current.

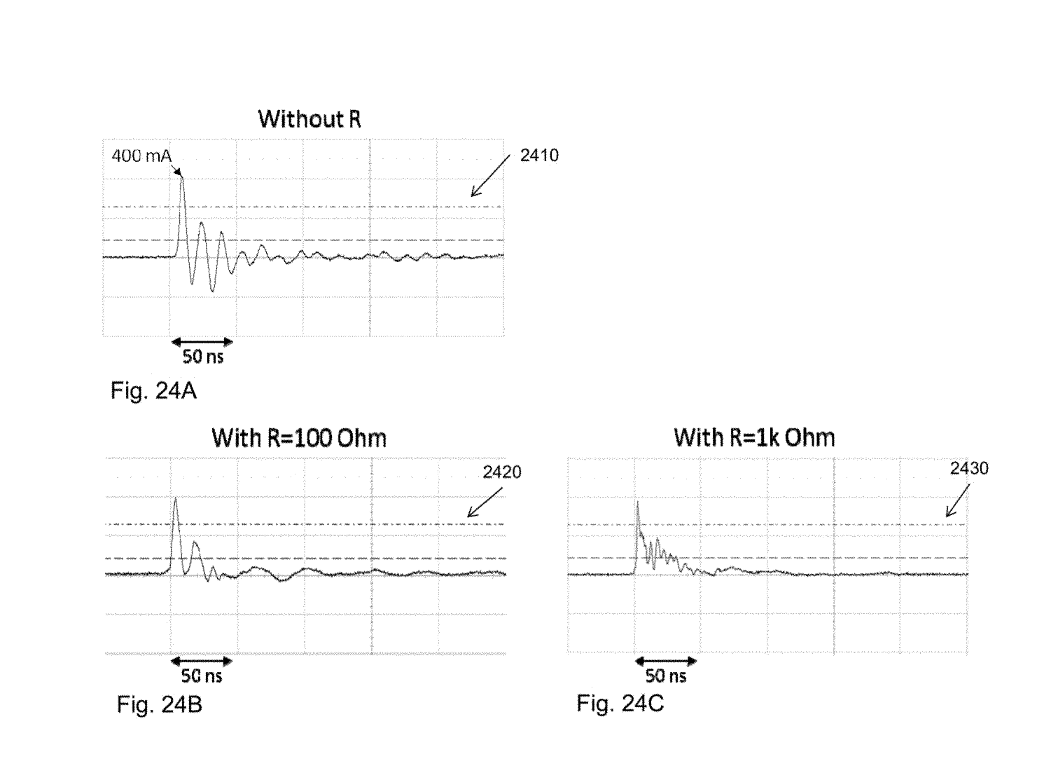

FIG. 24 shows current plots associated with exemplary resistor R values in circuitry 2325, where the applied voltage is an exemplary AC sinusoidal waveform. Changing the values of resistor R allow for temporally tuning the duration of the current peak, which controls the plasma duration on the skin 1620. In particular: FIG. 24A shows a current plot 2410 without a resistor (i.e., where R=0Ω); FIG. 24B shows a current plot 2420 with resistor R=100Ω; and FIG. 24C shows a current plot 2430 with resistor R=1 kΩ. Resistor R values may be chosen based on any particular application and any desired resistive characteristics. In some preferred embodiments, resistor R may be in a range between about 100Ω and about 1 GΩ.

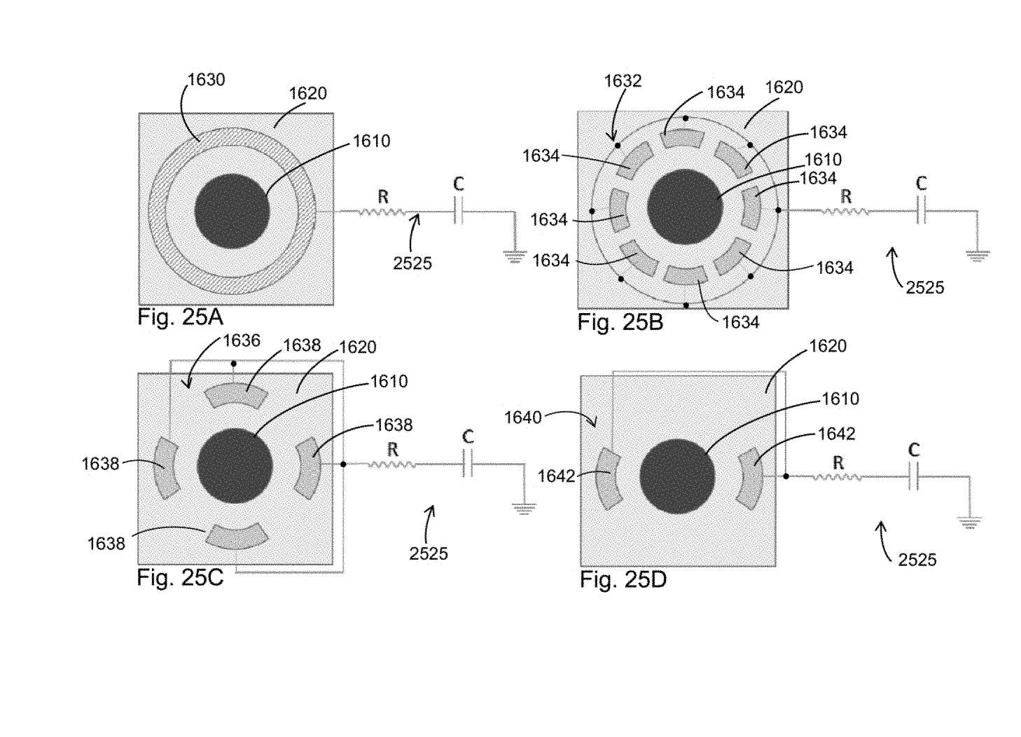

FIG. 25 shows various exemplary conductive elements connected to ground via a resistor R and a capacitor C. In these embodiments, the conductive elements in FIGS. 25A-25D and their respective configurations mimic those shown in FIGS. 16A-6D, respectively, but with circuitry 2525 including resistor R in series with capacitor C to ground. Introducing capacitor C between the conductive elements 1630, 1632, 1636, 1640 and the ground (with or without resistor R) can dynamically change the electric field and allow for temporally tuning the electric field in the skin 1620. For example, when the plasma is on, the capacitor C is charged to a certain voltage and when the plasma is off, the capacitor C is discharged. The discharge current after the plasma is off can be used to sustain the electric field in the skin 1620 for a period of time when the plasma is off. The value of the capacitor C can be such that the time constant of circuitry 2525 would enable the charging of the capacitor C when the plasma is on and discharging the capacitor C when the plasma is off. Resistor R in series with the capacitor C can limit the current flow during charging capacitor C to control the rate of charging, can limit the current flow from the discharging capacitor C, and can reduce the voltage that is seen by the conductive elements 1630, 1632, 1636, 1640. Circuitry 2525 with capacitor C also allows further tuning of the electric field around the conductive elements 1630, 1632, 1636, 1640 by inserting different values of capacitors.

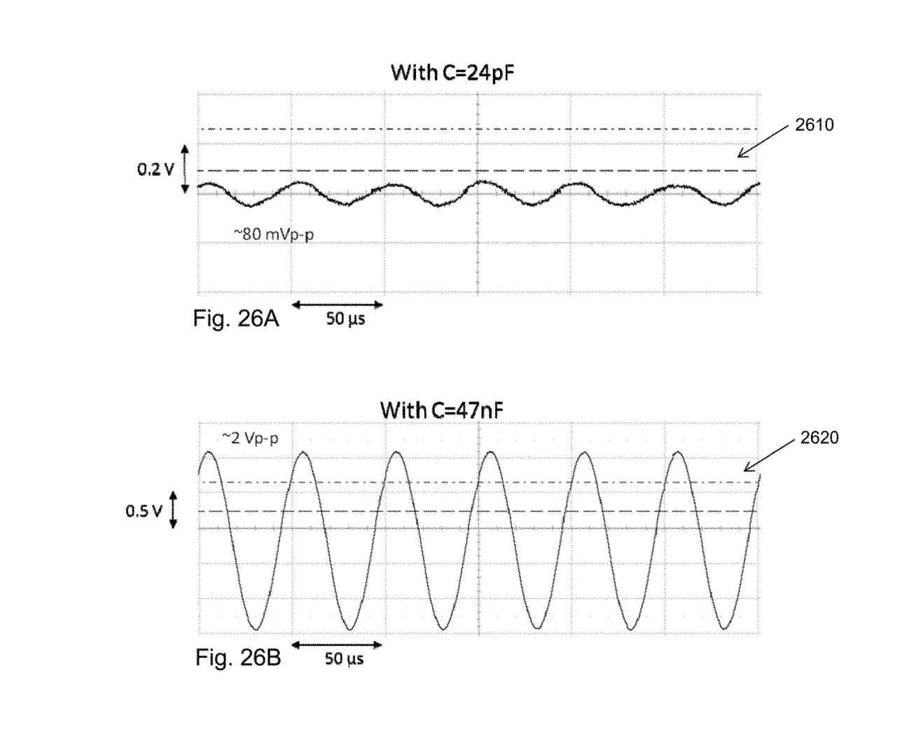

FIG. 26 shows voltage plots associated with exemplary capacitor C values in circuitry 2525, where the applied voltage is an exemplary AC sinusoidal waveform. Capacitor C allows for temporally tuning the electric field (voltage) around the conductive elements 1630, 1632, 1636, 1640 by using different values of capacitor C (and/or an inductor, as discussed below), which controls the electric field in the skin 1620. In particular: FIG. 26A shows a voltage plot 2610 with capacitor C=24 pF; and FIG. 26B shows a voltage plot 2620 with capacitor C=47 nF. In these embodiments resistor R values may be in a range between about 100Ω and about 10 kΩ. Capacitor C values may be chosen based on any particular application and any desired capacitive characteristics. In some preferred embodiments, capacitor C may be in a range between about 1 nF and about 100 μF. As mentioned above, resistor R values may also be chosen based on any particular application and any desired resistive characteristics. In some preferred embodiments, resistor R may be in a range between about 100Ω and about 1 GΩ.

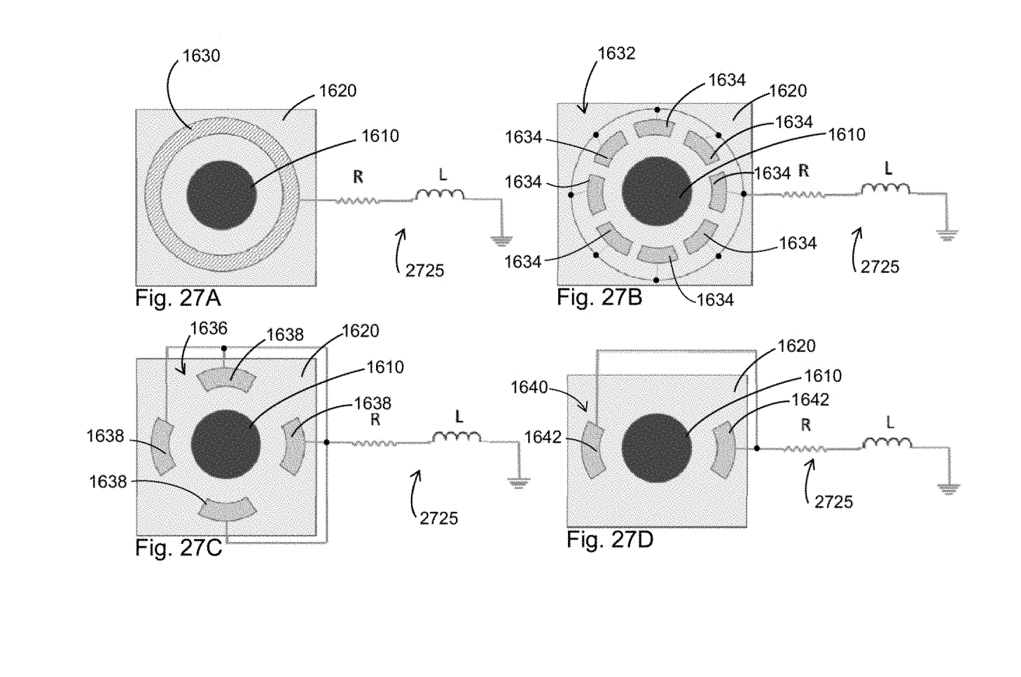

FIG. 27 shows various exemplary conductive elements connected to ground via a resistor R and an inductor L. In these embodiments, the conductive elements in FIGS. 27A-27D and their respective configurations mimic those shown in FIGS. 16A-6D, respectively, but with circuitry 2725 including resistor R in series with inductor L to ground. Introducing inductor L between the conductive elements 1630, 1632, 1636, 1640 and the ground (with or without resistor R) can dynamically change the electric field, for example, by dissipating the stored energy as heat, leading to enhanced skin permeation. For example, when the plasma is on, the inductor L provides a time varying bias to the skin 1620, further temporally tuning the electric field in the skin 1620 and when the plasma is off, the inductor L serves as a short circuit. The value of the inductor L can be such that the induced voltage would prevent the discharge current from reaching the sensation threshold. Circuitry 2725 with inductor L also allows further tuning of the electric field around the conductive elements 1630, 1632, 1636, 1640 by inserting different values of inductors. Inductor L values may be chosen based on any particular application and any desired inductive characteristics. In some preferred embodiments, inductor L may be in a range between about 1 μH and about 1 mH. As mentioned above, resistor R values may also be chosen based on any particular application and any desired resistive characteristics. In some preferred embodiments, resistor R may be in a range between about 100Ω and about 1 GΩ.

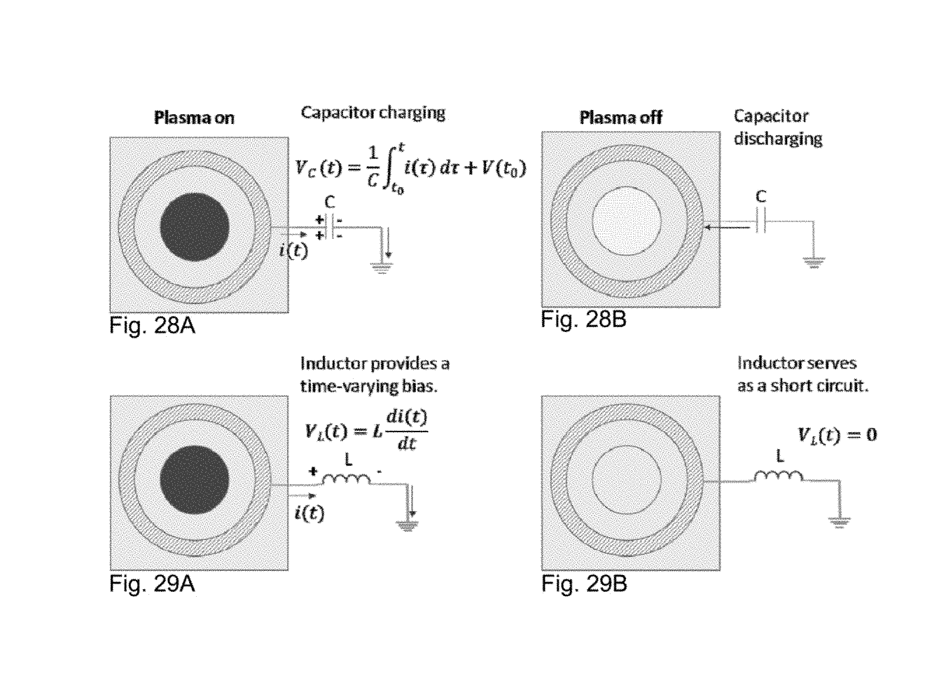

The use of capacitors C and/or inductors L that dynamically change the electric field provide time-varying characteristics that may be used to temporally tune the electric field in the skin. These time-varying characteristics are shown in FIGS. 28 and 29. In particular: FIG. 28A shows the capacitor C time-based voltage/current characteristics when the plasma is on; FIG. 28B shows the capacitor C time-based voltage/current characteristics when the plasma is off; FIG. 29A shows the inductor L time-based voltage/current characteristics when the plasma is on; and FIG. 29B shows the inductor L time-based voltage/current characteristics when the plasma is off.

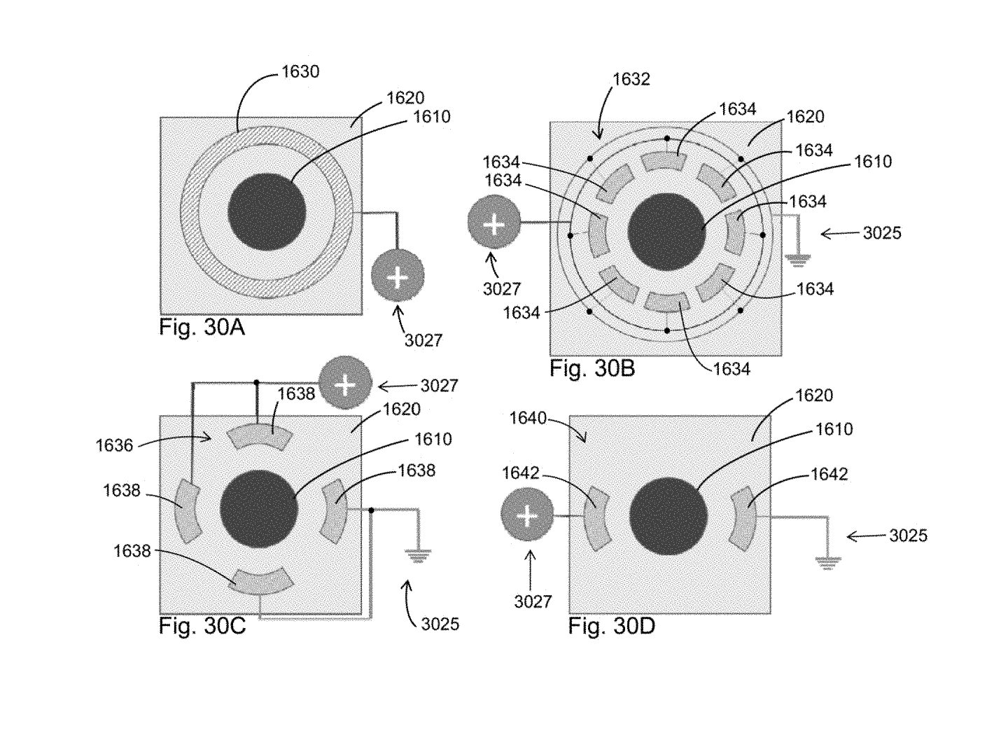

FIG. 30 shows various exemplary conductive elements connected to ground and/or a bias voltage. In these embodiments, the conductive elements in FIGS. 30A-30D and their respective configurations mimic those shown in FIGS. 16A-6D, respectively, but with circuitry 3025 connecting a subset of the conductive elements to ground and/or circuitry 3027 connecting another subset of the conductive elements to a bias voltage (+). Introducing bias voltage (+) to one or more of the conductive elements 1630, 1632, 1636, 1640 can dynamically change the electric field. For example, by modulating the value of the applied bias voltage (+), the electric field in the skin 1620 can be modulated spatially and temporally. Selectively connecting circuitry 3025 (ground) and/or circuitry 3027 (bias voltage (+)) allows further tuning of the electric field around the conductive elements 1630, 1632, 1636, 1640 by selecting which conductive elements 1630, 1632, 1636, 1640 to connect the circuitry 3025, 3027 to, by selecting different values of the bias voltage (+), and/or by modulating the values of the bias voltage (+). Bias voltage (+) values may be chosen based on any particular application and any desired voltage-difference characteristics.

It should be appreciated that the circuits 1625, 2325, 2525, 2725, 3025, 3027 discussed above are exemplary and in some cases simplified to demonstrate the particular characteristics of certain components. In other embodiments, various resistors, capacitors, inductors, bias voltages, and any other electrical components may be combined for use in other circuits, some very complex, including, for example, with combined characteristics, to meet the needs of any application.

In addition, the use of optional or variable components (e.g., variable resistors, capacitors, and inductors) may be used for providing an adjustable or variable skin treatment apparatus. Optional components may be selectively incorporated into a circuit or not. In some embodiments, banks of optional components may be used to provide a variety of individually selectable component values, which may also be combined. Variable components may be any type of component (e.g., digital or analog) that can adjust a characteristic (including, e.g., resistive, capacitive, inductive, etc.) of a circuit or signal. In this manner, the variable skin treatment apparatus can be tuned (by varying the value of the optional and/or variable components) for a particular application without the need for swapping fixed components or devices. In another embodiment, the variable skin treatment apparatus can be used for multiple applications that require different electric field characteristics without the need for changing devices. In some embodiments, a controller (e.g., controller 550 shown in FIG. 5) associated with a variable skin treatment apparatus can be used to automatically adjust the parameters of the variable skin treatment apparatus (including, e.g., optional and/or variable components) for different applications or skin treatments.

Furthermore, any transdermal delivery system can include one or more conductive elements, segments of the conductive elements, and various circuitry associated with the various conductive elements, segments of the conductive elements, and/or subsets thereof. For example, any of the circuitry 1625, 2325, 2525, 2725, 3025, 3027 can be connected to any of the conductive elements 1630, 1632, 1636, 1640, and/or subsets thereof (e.g., as shown in FIG. 30) in any number of combinations to tune and control the electric field in the skin spatially and/or temporally to achieve the desired results for any particular application. In one exemplary embodiment of a transdermal delivery system, one conductive element may be grounded, a first subset of segments of a second conductive element may be connected to circuitry with an inductor and a resistor, and a second subset of segments of the second conductive element may be connected to circuitry with a modulated bias voltage. In this manner, the combinations of conductive elements (including, for example, material, shape, number, segments, location, etc.), and circuits are unlimited. These features can also be combined with differences in other features of the transdermal delivery system, including, for example, power supplies, waveform circuitry, etc., to suit any application

In this manner, the transdermal delivery or skin treatment systems include several features, including, for example, conductive elements and associated circuitry, that provide for tuning and controlling the plasma-generated electric field directed selectively through regions of the skin. The electric field can be tuned spatially and temporally. The tuned characteristics can include various features of the electric field, including, for example, strength, depth, width, to selected regions of the skin, localization, etc., including time-varying and/or modulation. These characteristics can be used to control the desired poration in the selected regions of the skin. Poration can be intercellular, intracellular, or both.

Various skin treatments include a desired (targeted) poration of the skin to provide a desired rate of delivery of a selected substance (e.g., drugs, DNA, RNA, vaccines, proteins, molecules, macromolecules, etc.) after plasmaporation. Skin treatments can include topical, transdermal, and systemic deliveries and treatments, which can occur before, during, and/or after plasmaporation. Selective poration of the skin can target one or more regions of the skin and/or the surrounding tissue, including, for example, the stratum corneum, the epidermis, the blood stream, etc. Tuning and controlling the spatial and temporal electric field generated in skin due to plasmaporation can be used to control the depth and width of permeation of a topically applied substance. Additionally, focusing the electric field in a targeted region of the skin or tissue selectively porates only that region of the skin or tissue needed to deliver the drug, thus localizing its effect.

Accordingly, the ability to tune and control the electric field of a transdermal delivery or skin treatment system using conductive elements and associated circuitry can be combined with other plasma treatment parameters to control the delivery (e.g., depth of permeation) of a substance, including, for example, the type of plasma generator used, frequency, duty cycle, pulse duration, time of plasma treatment, time of application on the skin, etc.

Tuning and controlling the electric field of a transdermal delivery or skin treatment system may be used to drive common topical drugs into the skin faster. Advantages of delivering common topical drugs into the skin faster include, maintaining tighter therapeutic concentrations, eliminating the need for mixing the topical drugs with other compounds such as messy gels for proper absorption. The methodology may result in no need for additional FDA approval or increased speed of approval.

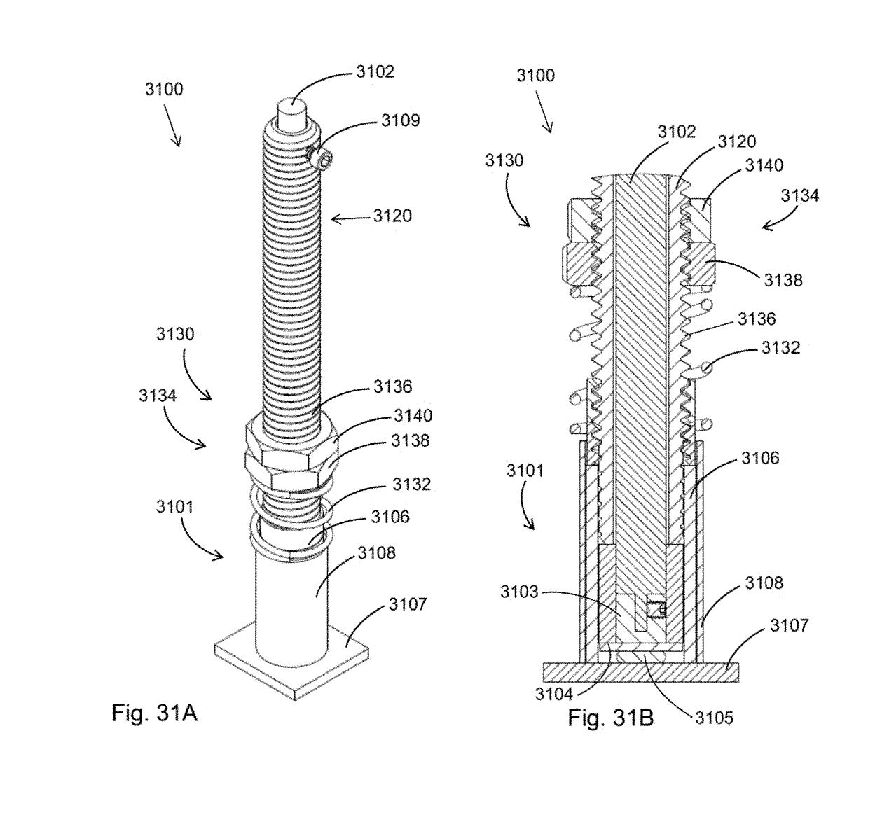

FIG. 31A is a drawing of an exemplary transdermal delivery apparatus 3100. FIG. 31B is a cross-section drawing of the lower portion of the apparatus 3100. Transdermal delivery apparatus 3100 includes a plasma generator 3101. Plasma generator 3101 includes a high voltage cable 3102 connected to an electrode 3103 on a first end and a high voltage power supply (not shown) on the second end. The power supply and its associated circuitry and waveforms can be any of those mentioned above. The plasma generator 3101 is a non-thermal DBD generator with a dielectric barrier 3104 located below the high voltage electrode 3103. Plasma 3105 can be generated between the dielectric barrier 3104 and the skin 3107. The high voltage electrode 3103 and the dielectric barrier 3104 can be located within a housing 3106, along with additional components, as discussed above.

Plasma 3105 is generated by the plasma generator 3101 above skin 3107 and can be in direct contact with the skin 3107. In this embodiment, the apparatus 3100 also incorporates an exemplary conductive element 3108 that is also in contact with the skin 3107. In this embodiment, the conductive element 3108 is cylindrical with a circular shape and surrounds the generated plasma 3105. Also in this embodiment, a strain relief screw 3109 is shown for securing the high voltage cable 3102 within body 3120. Circuitry (not shown, described above) associated with the conductive element 3108 may be placed in circuit communication with the conductive element 3108 via a conductive tab (not shown). In another embodiment, circuitry may be integrated with the apparatus 3100. In another embodiment, a floating ground may be utilized by not connecting circuitry or a ground to the conductive element 3108. The plasma 3105 and conductive element 3108 direct the electric field associated with the plasma 3105 through at least a region of the skin 3107.

In this embodiment, the apparatus 3100 also includes a spring-loaded mechanism 3130 with a spring 3132 that biases the conductive element 3108 in a non-ready position until the conductive element 3108 is in proper contact with the skin 3107. In operation, the apparatus 3100 is placed in contact with the skin 3107 with applied pressure such that the spring 3132 of the spring-loaded mechanism 3130 compresses to ensure that the conductive element 3108 is in contact with the skin 3107. In some embodiments, the apparatus 3100 includes a position sensing device (not shown) that changes state when the conductive element 3108 contacts the skin 3107 and compresses the spring 3132 of the spring-loaded mechanism 3130. For example, in one embodiment, the sensing device is a switch that closes when the conductive element 3108 is in contact with the skin 3107, indicating that the apparatus 3100 is in a ready position, as discussed in detail above. In this embodiment, the spring-loaded mechanism 3130 also includes an adjustable tension mechanism 3134. In particular, the adjustable tension mechanism 3134 includes a threaded shaft 3136 on the conductive body 3120 with a corresponding nut 3138 (and optional locking nut 3140) that forms a stop against the spring 3132. In some embodiments, the spring-loaded mechanism 3130 can also be used to determine a distance between the electrode 3103 and the skin 3107, including, for example, by use of a mechanical stop (not shown). In other embodiments, other tension-control or biasing mechanisms may be used as equivalents to the spring-loaded mechanism 3130 and adjustable tension mechanism 3134 for the same purpose.

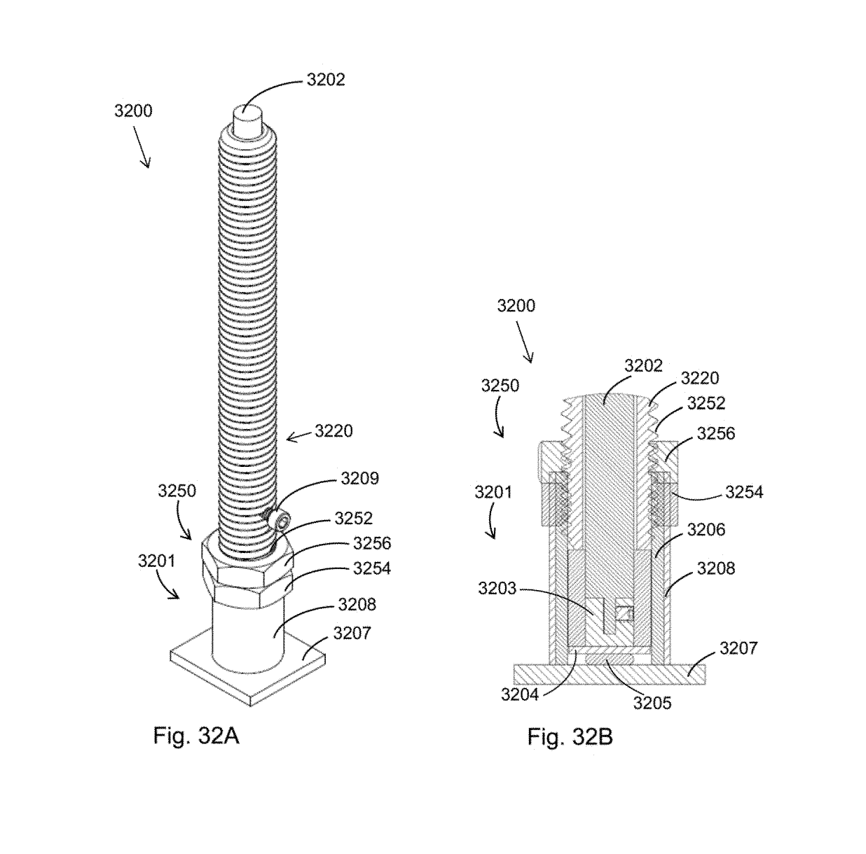

FIG. 32A is a drawing of another exemplary transdermal delivery apparatus 3200. FIG. 32B is a cross-section drawing of the lower portion of the apparatus 3200. Transdermal delivery apparatus 3200 includes a plasma generator 3201. Plasma generator 3201 includes a high voltage cable 3202 connected to an electrode 3203. The plasma generator 3201 is a non-thermal DBD generator with a dielectric barrier 3204 located below the high voltage electrode 3203. Plasma 3205 can be generated between the dielectric barrier 3204 and the skin 3207. The high voltage electrode 3203 and the dielectric barrier 3204 can be located within a housing 3206, along with additional components, as discussed above.

In this embodiment, the apparatus 3200 also incorporates an exemplary conductive element 3208 that surrounds the generated plasma 3205. A strain relief screw 3209 is shown for securing the high voltage cable 3202 within body 3220. Circuitry (not shown, described above) associated with the conductive element 3208 may be placed in circuit communication with the conductive element 3208 via a conductive tab (not shown). The plasma 3205 and conductive element 3208 direct the electric field associated with the plasma 3205 through at least a region of the skin 3207.

In this embodiment, the apparatus 3200 also includes an adjustable height mechanism 3250. In particular, the adjustable height mechanism 3250 includes a threaded shaft 3252 on the conductive body 3220 with a corresponding nut 3254 (and optional locking nut 3256) that forms a stop against the conductive element 3208. In this manner, the adjustable height mechanism 3250 is used to determine a distance between the electrode 3203 and the skin 3207 when the conductive element 3208 is placed in contact with the skin 3207.

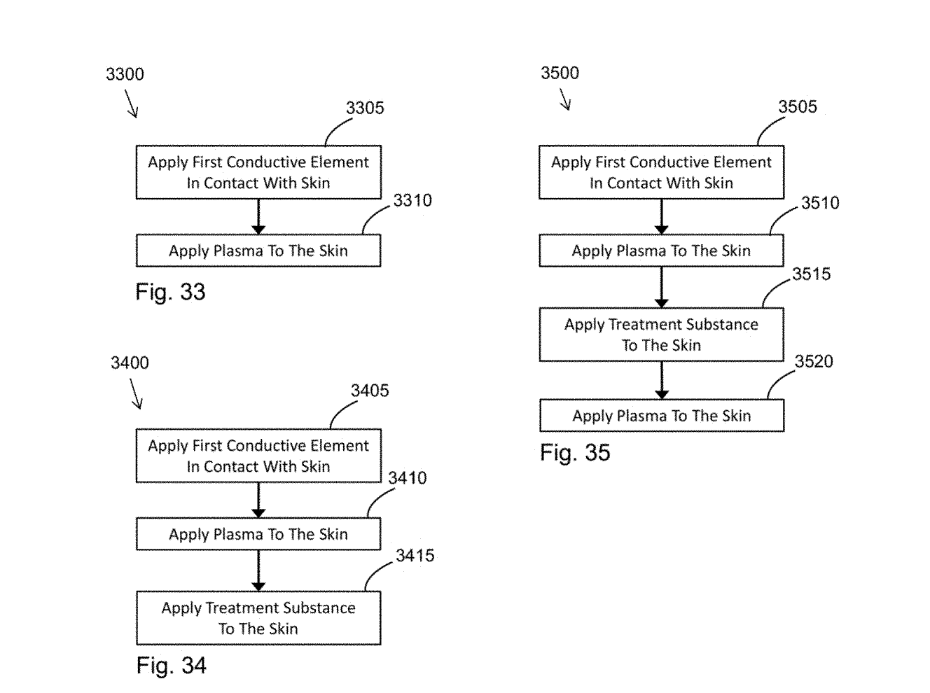

FIGS. 33-38 are block diagrams of exemplary methodologies associated with the skin treatment apparatus. The exemplary methodologies may be carried out in logic, software, hardware, or combinations thereof. In addition, although the methods are presented in an order, the blocks may be performed in different orders. Further, additional steps or fewer steps may be used.

FIG. 33 shows an exemplary method 3300 of treating skin using any of the systems, components, and/or configurations described above. First, at step 3305, the method includes applying a conductive element in contact with the skin. Then, at step 3310, the method includes applying a plasma to the skin. In this method, as described above, the conductive element affects the electric field associated with the plasma and at least a portion of an electric field is directed through a region of the skin by the plasma and the conductive element, causing poration in the skin.

FIG. 34 shows another exemplary method 3400 of treating skin using any of the systems, components, and/or configurations described above. The first two steps, 3405 and 3410, are the same as steps 3305 and 3310 of method 3300. At step 3415, the method includes applying a treatment substance to the skin after applying the plasma to the skin. FIG. 35 shows another exemplary method 3500 of treating skin using any of the systems, components, and/or configurations described above. The first three steps, 3505 through 3515, are the same as steps 3405 and 3415 of method 3400. At step 3520, the method includes applying the plasma to the skin after applying the treatment substance to the skin. The skin treatment methods 3400 and 3500 can increase the speed of permeation of a treatment substance into the skin.

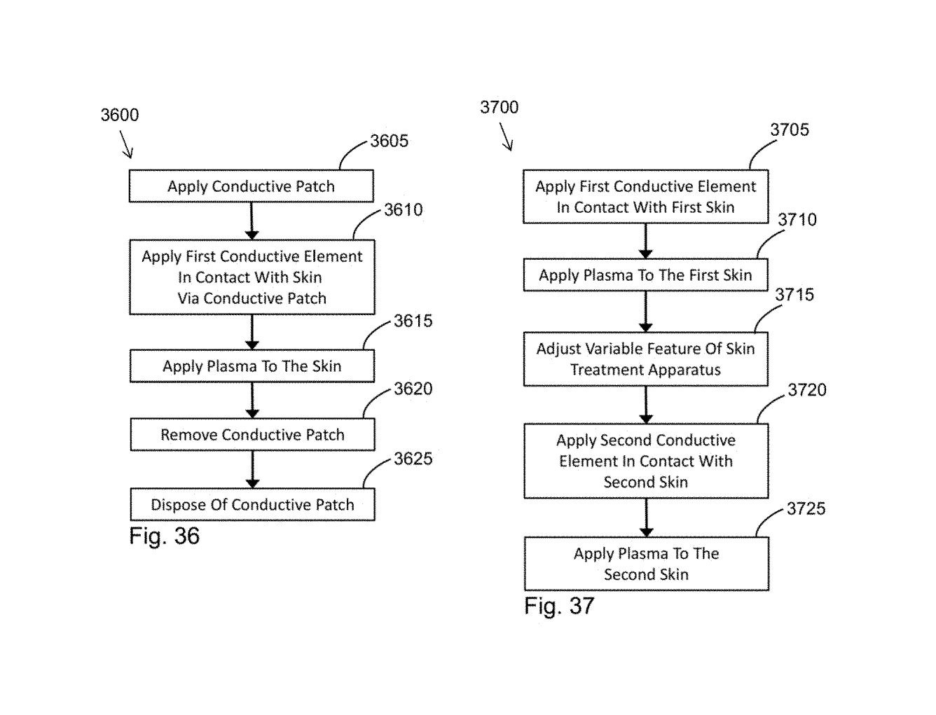

FIG. 36 shows another exemplary method 3600 of treating skin using any of the systems, components, and/or configurations described above. First, at step 3605, the method includes applying a conductive patch between the skin and the first conductive element. In one embodiment, the conductive patch is applied to the skin. In another embodiment, the conductive patch is applied to the conductive element. Next, at step 3610, the method includes applying the conductive element in contact with the skin via the conductive patch. Then, at step 3615, the method includes applying a plasma to the skin. Next, at step 3620, the method includes removing the conductive patch after the skin treatment. Finally, at step 3625, the method includes disposing the conductive patch after removing the conductive patch. In one embodiment, disposal includes discarding the conductive patch. In another embodiment, disposal includes cleaning or sterilizing the used conductive patch before reuse.

FIG. 37 shows an exemplary method 3700 of treating skin using a variable skin treatment apparatus adaptable for a plurality of skin treatments, and including any applicable systems, components, and/or configurations described above. First, at step 3705, the method includes applying a first conductive element in contact with a first skin. Then, at step 3710, the method includes applying a first plasma to the first skin using the variable skin treatment apparatus. In this method, as described above, the first conductive element affects a first electric field associated with the first plasma, and the first electric field is directed through the first skin by the first plasma and the first conductive element. Next, at step 3715, the method includes adjusting at least one variable feature of the variable skin treatment apparatus. Continuing, at step 3720, the method includes applying a second conductive element in contact with a second skin. Next, at step 3725, the method includes applying a second plasma to the second skin using the variable skin treatment apparatus. In this method, as described above, the second conductive element affects a second electric field associated with the second plasma, and the second electric field is directed through the second skin by the second plasma and the second conductive element. In this manner, the same skin treatment apparatus can be used for multiple skin treatment applications by adjusting one or more parameters of the treatment apparatus for each application, thus avoiding dedicated systems for each application.

In one embodiment of method 3700, the variable feature of the treatment apparatus is associated with the plasma generating device of the treatment apparatus. In another embodiment of method 3700, the variable feature of the treatment apparatus is associated with the power supply of the treatment apparatus. In another embodiment of method 3700, the variable feature of the treatment apparatus is associated with the circuitry for providing electrical pulses to the plasma generating device of the treatment apparatus. In another embodiment of method 3700, the variable feature of the treatment apparatus is associated with circuitry associated with the first conductive element, such as, for example, a variable resistor, a variable capacitor, and/or a variable inductor.

In another embodiment of method 3700, the first conductive element and the second conductive element are the same.

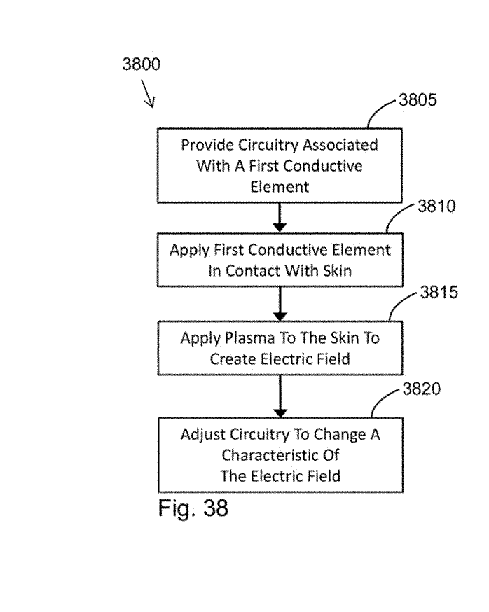

FIG. 38 shows an exemplary method 3800 of tuning a skin treatment apparatus using any of the systems, components, and/or configurations described above. First, at step 3805, the method includes providing circuitry associated with a conductive element. Next, at step 3810, the method includes applying the conductive element in contact with a skin. Then, at step 3815, the method includes applying a plasma to the skin using the skin treatment apparatus. In this method, as described above, at least a portion of an electric field is directed through the skin by the plasma and the conductive element, and the circuitry affects the electric field associated with the plasma. Finally, at step 3820, the method includes adjusting the circuitry to change a characteristic of the electric field.

In one embodiment of method 3800, the changed characteristic includes a spatial characteristic of the electric field. In another embodiment of method 3800, the changed characteristic includes a temporal characteristic of the electric field.

In other embodiments, any of the above methods can include the step of selecting the appropriate conductive element for a particular application, along with selecting any other device choice, parameter settings, substance choice, etc.

Although the embodiments described herein are described with respect to skin, the inventive concepts described herein are applicable to other tissue or organs. In addition, while certain substances (e.g., molecules, drugs, and vaccines) have been mentioned, the exemplary systems and methods described herein are applicable to many other substances, including, for example, DNA vaccines, to application of growth factors, antitumor drugs, chemotherapeutic drugs, immunomodulating drugs, particles and the like where it may be desirable to move the substance or item between cells, such as those in the stratum corneum and/or into cells, such as those in the epidermis or dermis.

Although many of the exemplary methods above relate to molecules, particles having similar molecular weights or equivalent diameters may also be transported across layers of the skin. In some embodiments, nanoparticles, such as, for example, silver nanoparticles, silver ions and other metal or polymer nanoparticles are driven into pores in the skin where they are allowed to react. Silver, copper and other metals are known to induce cell lysis and inhibit cell transduction. The introduction of silver and other metals in the form of nanoparticles increases the surface area available to react with microorganisms and enhances the antimicrobial action. Additionally, introduction of nanoparticles that encapsulate the molecule, vaccine, or drug of interest after plasmaporation allows permeation of such molecules to a controlled depth leading to controlled long term release of actives within a particular area of skin. Nanoparticles, including quantum dots, nanotubes and the like, having a diameter of between about 2 and about 400 nanometers may be driven across the skin using plasmaporation.

While the exemplary embodiments are illustrated using skin, any of the described embodiments would work equally well with any tissue, including, for example, epithelial tissue; mucosal epithelial tissue; muscle tissue; connective tissue; and inner and outer lining of organs.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. Therefore, the invention, in its broader aspects, is not limited to the specific details, the representative apparatus and methods, and illustrative examples shown and described. Accordingly, departures may be made from such details without departing from the spirit or scope of the applicant's general inventive concept. While the embodiments discussed herein have been related to the systems and methods discussed above, these embodiments are intended to be exemplary and are not intended to limit the applicability of these embodiments to only those discussions set forth herein. The control systems and methodologies discussed herein may be equally applicable to, and can be utilized in, other systems and methods.

We claim:

1. An apparatus for controlling an electric field associated with a skin treatment, comprising:
a plasma generating device for generating a plasma;
a power supply for powering the plasma generating device;
first circuitry for providing one or more first electrical pulses to the plasma generating device;
a first conductive element capable of placement in contact with the skin for affecting the electric field associated with the generated plasma;
wherein at least a portion of the electric field is directed through a first region of the skin by the plasma and the first conductive element.

2. The apparatus of claim 1, wherein the first conductive element is placed on the skin.

3. The apparatus of claim 1, wherein the first conductive element contacts the skin via a spring-loaded mechanism.

4. The apparatus of claim 3, wherein the spring-loaded mechanism comprises a position sensing device that changes state when the first conductive element contacts the skin and compresses a spring of the spring-loaded mechanism.

5. The apparatus of claim 3, wherein the spring-loaded mechanism comprises an adjustable tension mechanism.

6. The apparatus of claim 5, wherein the adjustable tension mechanism determines a distance between the plasma generating device and the skin.

7. The apparatus of claim 1, wherein the first conductive element contacts the skin via an adjustable height mechanism.

8. The apparatus of claim 1, wherein the first conductive element affects the electric field spatially.

9. The apparatus of claim 1, wherein the first conductive element affects the electric field temporally.

10. The apparatus of claim 1, wherein the first conductive element is grounded.

11. The apparatus of claim 1, wherein the first conductive element is operatively connected to second circuitry.

12. The apparatus of claim 11, wherein the second circuitry is configured to tune the characteristics of the electric field.

13. The apparatus of claim 12, wherein the electric field is spatially tuned to be directed to the first region of the skin.

14. The apparatus of claim 1, wherein the first conductive element comprises a plurality of segmented conductive elements.

15. The apparatus of claim 14, wherein the plurality of segmented conductive elements surround the plasma.

16. A method of treating skin comprising:
applying a first conductive element in contact with the skin; and
applying a plasma to the skin, wherein the first conductive element affects the electric field associated with the plasma;
wherein at least a portion of an electric field is directed through a first region of the skin by the plasma and the first conductive element.

17. The method of claim 16, wherein the electric field causes poration in the first region of the skin.

18. The method of claim 16, further comprising applying a treatment substance to the skin after applying the plasma to the skin.

19. The method of claim 18, further comprising applying the plasma to the skin after applying the treatment substance to the skin.

20. A method of tuning a skin treatment apparatus, comprising:
providing circuitry associated with a first conductive element;
applying the first conductive element in contact with a skin;
applying a plasma to the skin using the skin treatment apparatus, wherein at least a portion of an electric field is directed through the skin by the plasma and the first conductive element;
wherein the circuitry affects the electric field associated with the plasma; and
adjusting the circuitry to change a characteristic of the electric field.
